(19) United States
(12) Patent Application Publication
(10) Pub. No.: US 2004/0102473 A1
(43) Pub. Date: May 27, 2004

(54) Method of preventing or treating atherosclerosis or restenosis

(76) Inventors: Michael W. Wathen, Thousand Oaks, CA (US); Lynne K. Wathen, Thousand Oaks, CA (US)

Correspondence Address: FLYNN, THIEL, BOUTELL & TANIS, P.C., 2026 RAMBLING ROAD, KALAMAZOO, MI, 49008-1699, US

(21) Appl. No.: 10/651,216

(22) Filed: Aug. 28, 2003

Related U.S. Application Data
(60) Provisional application No. 60/407,090, filed on Aug. 30, 2002.

(51) Int. Cl. A61K 31/4743

(52) U.S. Cl. 514/301

(57) ABSTRACT

The present invention provides a method of treating atherosclerosis or restenosis in a mammal which comprises administering to said mammal an effective amount of a compound selected from the group consisting of structures of Formulae I, I′ and II,

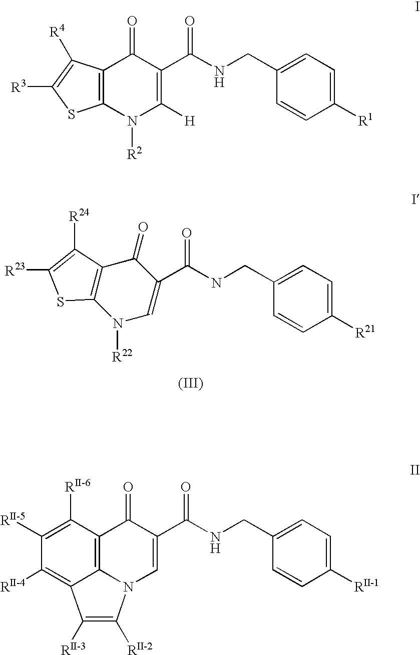

wherein the substituents on the Formulae are as defined herein.

### CROSS-REFERENCE TO RELATED APPLICATIONS

This application claims the benefit of U.S. provisional application Serial No. 60/407 090, filed Aug. 30, 2002, under 35 USC 119(e)(i), which is incorporated herein by reference.

### BACKGROUND OF THE INVENTION

This invention relates to a method of preventing or treating atherosclerosis and restenosis in mammals.

Atherosclerosis is characterized by the deposition of fatty substances in and fibrosis of the inner layer of the arteries. Restenosis is an accelerated form of atherosclerosis that commonly occurs after angioplasty surgery and atherectomy.

Cardiovascular diseases (CVD) contribute substantially to illness and death worldwide and ranks second only to infectious and parasitic diseases as human affliction. Atherosclerosis, a major component of CVD, has properly been considered a public health problem of industrialized countries, accounting for an estimated one third of deaths overall. It has been reported that in the United States alone, atherosclerosis affects one in four persons, causing approximately 42% of all deaths. O'Connor et al, “Potential Infectious Etiologies of Atherosclerosis: A Multifactorial Perspective”, Emerging Infectious Disease, Vol. 7, No. 5, September-October 2001.

It has been suggested that the number of chronic infective pathogens which an individual has been exposed independently contribute to the long-term prognosis in patients with documented coronary artery disease. H J Rupprecht et al, “Impact of Viral and Bacterial Infective Burden on Long-term Prognosis in Patients with Coronary Artery Disease. (Circulation (2001) 104:25-31. Seropositivity to multiple herpesviruses is an independent risk factor for death from cardiovascular disease and risk is proportional to the number of different herpesviruses that have infected an individual. Other investigators that have suggested a connection between infectious pathogens and atherosclerosis include Espinola-Klein et al, “Impact of Infectious Burden on Extent and Long-Term Prognosis of Atherosclerosis”, Circulation (2002) 105:15-21; O'Connor et al, Supra: and Zhou et al, “Association Between Prior Cytomegalovirus Infection and the Risk of Restenosis after Coronary Atherectomy”, The New England Journal of Medicine (1996). An antiviral drug, Ganciclovir, has been shown to prevent atherosclerosis resulting from CMV infection of rats (K. B. Lemstrom et al. Cytomegalovirus infection-enhanced allograft arteriosclerosis is prevented by DHPG prophylaxis in the rat. Circulation, 1994,90:1969-1978).

Herpesviruses are believed to be a particular problem in atherosclerosis because they reside latently in an infected individual and can reactivate causing a chronic inflammatory response. The herpesvirus family contains eight known human viruses; herpes simplex virus type 1 (HSV-1), herpes simplex virus type 2 (HSV-2), varicella zoster virus (VZV), human cytomegalovirus (HCMV), human herpes virus 6 (HHV-6), human herpes virus 7 (HHV-7), Epstein-barr virus (EBV) and human herpes virus 8 (HHV-8). One of the hallmarks of herpesviruses is their ability to establish latent infections in their host and to recur during times of stress or immunosuppression. The human herpesviruses are associated with a diverse set of diseases ranging in severity from mild cold sores to life-threatening illness in immunocompromised patients (Table 1).

TABLE 1
Herpesvirus diseases and treatment

| | Associated Diseases | | |
|---|---|---|---|
| | | Immunocompromised | Marketed |
| Virus | Normal Host | Host | Antivirals |
| HSV-1 | Herpes labialis | Disseminated herpes | • Acyclovir |
| | (cold sores) | | • Penciclovir |
| HSV-2 | Genital herpes | Disseminated herpes | • Acyclovir |
| | | | • Valaciclovir |
| | | | • Famciclovir |
| VZV | Chicken pox | Herpes zoster | • Acyclovir |
| | Herpes zoster | | • Valaciclovir |
| | | | • Famciclovir |
| CMV | Congenital CMV | Retinitis | • Ganciclovir |
| | disease | Pneumonia | • Valganciclovir |
| | | GI disease | • Foscarnet |
| | | Graft rejection | • Cidofovir |
| | | | • Formivirsen |
| EBV | Infectious | Lymphomas (PTLD) | • None |
| | mononucleosis | | |
| HHV-6 | Exanthem subitum | Graft rejection | • None |
| HHV-7 | Exanthem subitum | Graft rejection | • None |
| HHV-8 | Kaposi's sarcoma | Kaposi's sarcoma | • None |

HSV-1, HCMV, VZV and EBV are ubiquitous viruses with seroprevalence rates in adults of 70-80% for HSV-1 and 90-100% for HCMV, VZV and EBV. Seroprevalence of HSV-2 increases from about 10% in young adults to 35% by age 60. Antibodies to HHV-8 are also found in about 33% of adults in the United States. The high seroprevalence of multiple viruses and their ability to reactivate from latent infections, make these herpesviruses prime candidates for causing chronic inflammatory responses leading to atherosclerosis.

Numerous studies and articles on the epidemiology of the herpesvirus family are in the prior art. Wathen, Michael W., “Non-nucleoside inhibitor of herpesviruses”, Rev. Med. Virol, 2002; 12: 167-178; Whitley et al, “Herpes Simplex Viruses”, Clinical Infection Diseases, 1998; 26: 541-55, Cohen, Jeffrey I., “Epstein-Barr Virus Infection”, Medical Progress, Volume 343, Number 7, The New England Journal of Medicine, Aug. 17, 2000, pp. 481-492; Blouvelt et al; “Human Herpes Virus 8 Infection Occurs Following Adolescence in the United States”, The Journal of Infectious Disease, 1997, 176: 771-4; Field, A. Kirk, “Human Cytomegalovirus: challenge opportunities and new drug development”, Antiviral Chemistry and Chemotherapy 10: 219-232.

### INFORMATION DISCLOSURE

U.S. Pat. No. 6,239,142 discloses 4-oxo-4,7-dihydro-thieno[2,3-b]pyridine-5-carboxamide derivatives, compounds of Formula I and I′ that are useful as antiviral agents. These compounds have now been found to be useful in the method of this invention.

U.S. Pat. No. 6,291,437 describes a method for preventing or retarding the development of atherosclerotic lesions or restenosis comprising administering to a subject, preferably a human, an effective amount of an anti-viral composition directed against CMV, and optionally anti-microbial composition directed against C. pneumoniae.

WO 02/48148 A2 discloses anti-viral compounds and a method of using them for the prophylaxis or treatment of atherosclerosis, coronary artery disease or restenosis.

An antiviral drug, Ganciclovir, has been shown to prevent atherosclerosis resulting from CMV infection of rats (K. B. Lemstrom et al. Cytomegalovirus infection-enhanced allograft arteriosclerosis is prevented by DHPG prophylaxis in the rat. Circulation, 1994,90:1969-1978).

U.S. Pat. No. 6,239,142 disclosed compounds and their use to treat herpesvirus infections.

WO 02/06513 disclosed method of screening 4-hydroxyquinline, 4-oxo-dihydroquinoline, and 4-oxo-dihydrothienopyridine derivatives as non-nucleoside herpesvirus DNA polymerase inhibitors.

EP 443568 disclosed fused thiophene derivatives, their production and use.

WO 02/04445 disclosed a variety of tricyclic core structures which have antiviral activity against herpesviruses.

WO 02/04444, WO 02/04443, and WO 02/04422 disclosed a variety of bicyclic core structures which have antiviral activity against herpesviruses.

U.S. Pat. No. 6,248,739 disclosed compounds in which the core structure is a quinoline and useful as antivirals against herpesviruses.

### OBJECT OF THE INVENTION

It is the object of this invention to provide a method for preventing or treating atherosclerosis or restenosis in mammals utilizing compounds of Formulae I, I′ and II.

It is a further objective of this invention to provide a method for prophylaxis of atherosclerosis and treat patients who have atherosclerosis utilizing compounds of Formula I, I′ and II.

It is still a further objective of the invention to provide a method that prevents or ameliorates the occurrence of restenosis in patients anticipating coronary atheroscopy or angioplasty, utilizing compounds of Formulae I, I′ and II.

### SUMMARY OF THE INVENTION

This invention provides a method of preventing or treating atherosclerosis or restenosis in a mammal, comprising administering to said mammal an effective amount of the compound selected from the group consisting of structures of Formula I, Formula I′ and Formula II; wherein Formula I is

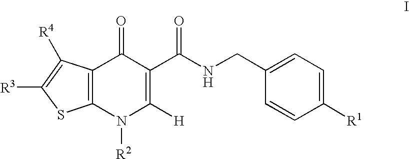

or a pharmaceutically acceptable salt, racemate, solvate, tautomer, optical isomer or prodrug derivative thereof, wherein,

R¹ is

(a) Cl,

(b) Br,

(c) CN,

(d) NO₂, or

(e) F;

R² is

(a) H,

(b) R⁵,

(c) NR⁷R⁸,

(d) SO₂R⁹, or

(e) OR⁹;

R³ is

(a) H,

(b) halo,

(c) aryl,

(d) S(O)ₘR⁶,

(e) (C═O)R⁶,

(f) (C═O)OR⁹,

(g) cyano,

(h) het, wherein said het is bound via a carbon atom,

(i) OR¹⁰,

(j) Ohet,

(k) NR⁷R⁸

(l) SR₁₀,

(m) Shet,

(n) NHCOR¹²,

(o) NHSO₂R¹², or

(p) C₁₋₇alkyl which may be partially unsaturated and optionally substituted by one or more substituents of the group R¹¹, OR¹³, SR¹⁰, SR¹³, NR⁷R⁸, halo, (C═O)C₁₋₇alkyl, or SOₘR⁹;

R⁴ is

(a) H,

(b) halo,

(c) C₁₋₄alkyl, or

(d) R⁴ together with R³ form a carbocyclic or het, either of which may be optionally substituted by NR⁷R⁸, by C₁₋₇alkyl which may be optionally substituted by OR¹⁴, or by het, wherein said het is bound via a carbon atom;

R⁵ is

(a) (CH₂CH₂O)ᵢR¹⁰,

(b) het, wherein said het is bound via a carbon atom,

(c) aryl,

(d) C₁₋₇alkyl which may be partially unsaturated and is optionally substituted by one or more substituents selected from a group consisting of NR⁷R⁸, R¹¹, SOₘR⁹, and OC₂₋₄alkyl which may be further substituted by het, OR¹⁰, or NR⁷R⁸, or

(e) C₃₋₈cycloalkyl which may be partially unsaturated and optionally substituted by one or more substituents selected from a group consisting of R¹¹, NR⁷R⁸, SOₘR⁹, and C₁₋₇alkyl optionally substituted by R¹¹, NR⁷R⁸, or SOₘR⁹;

R⁶ is

(a) C₁₋₇alkyl,

(b) NR⁷R⁸,

(c) aryl, or

(d) het, wherein said het is bound via a carbon atom;

R⁷ and R⁸ are independently

(a) H,

(b) aryl,

(c) C₁₋₇alkyl which may be partially unsaturated and is optionally substituted by one or more substituents selected from a group consisting of NR¹⁰R¹⁰, R¹¹, SOₘR⁹, CONR¹⁰R¹⁰, or halo, or,

(d) R⁷ and R⁸ together with the nitrogen to which they are attached form a het;

R⁹ is

(a) aryl,

(b) het,

(c) C₃₋₈cycloalkyl, or

(d) C₁₋₇alkyl which may be partially unsaturated and is optionally substituted by one or more substituents selected from a group consisting of NR¹⁰R¹⁰, R¹¹, SH, CONR¹⁰R¹⁰, or halo;

R¹⁰ is

(a) H, or

(b) C₁₋₇alkyl optionally substituted by OH;

R¹¹ is

(a) OR¹⁰,

(b) Ohet,

(c) Oaryl,

(d) CO₂R¹⁰,

(e) het,

(f) aryl, or

(g) CN;

R¹² is

(a) H,

(b) het,

(c) aryl,

(d) C₃₋₈cycloalkyl, or

(e) C₁₋₇alkyl optionally substituted by NR⁷R⁸ or R¹¹;

R¹³ is

(a) (P═O)(OR¹⁴)₂,

(b) CO(CH₂)ₙCON(CH₃)—(CH₂)ₙSO₃⁻M⁺,

(c) an amino acid,

(d) C(═O)aryl, or

(e) C(═O)C₁₋₇alkyl optionally substituted by NR⁷R⁸, aryl, het, CO₂H, or O(CH₂)ₙCO₂R¹⁴);

R¹⁴ is

(a) H, or

(b) C₁₋₇alkyl;

each i is independently 2, 3, or 4;

each n is independently 1, 2, 3, 4 or 5;

each m is independently 0, 1, or 2; and

M is sodium, potassium, or lithium;

wherein any aryl is optionally substituted with one or more substituents selected from the group consisting of halo, OH, cyano, CO₂R¹⁴, CF₃, C₁₋₆alkoxy, and C₁₋₆ alkyl which maybe further substituted by one to three SR¹⁴, NR¹⁴R¹⁴, OR¹⁴, het, and CO₂R¹⁴; and

wherein any het is optionally substituted with one or more substituents selected from the group consisting of halo, OH, cyano, phenyl, CO₂R¹⁴, CF₃, C₁₋₆alkoxy, oxo, oxime, and C₁₋₆ alkyl which maybe further substituted by one to three SR¹⁴, NR¹⁴R¹⁴, OR¹⁴, and CO₂R¹⁴;

aryl, in Formula I, denotes a phenyl radical or an ortho-fused bicyclic carbocyclic radical having about nine to ten ring atoms in which at least one ring is aromatic. Het is a four-(4), five-(5), six-(6), or seven-(7) membered saturated or unsaturated heterocyclic ring having 1, 2, 3, or 4 heteroatoms selected from the group consisting of oxy, thio, sulfinyl, sulfonyl, and nitrogen, which is optionally fused to a benzene ring, or any bicyclic heterocycle group;

het, in Formula I, includes “heteroaryl,” which encompasses a radical attached via a ring carbon of a monocyclic aromatic ring containing five or six ring atoms consisting of carbon and 1, 2, 3, or 4 heteroatoms each selected from the group consisting of non-peroxide oxy, thio, and N(X) wherein X is absent or is H, O, C₁₋₄alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto; and wherein Formula I′ is

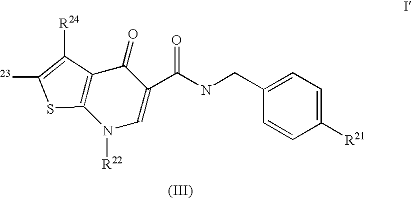

or a pharmaceutically acceptable salt, racemate, solvate, tautomer, optical isomer or prodrug derivative thereof wherein,

R²¹ is Cl, Br, CN, or NO₂;

R²² is H, —(CH₂CH₂O)ₙH, —(CH₂CH₂O)ₙCH₃, SO₂R³⁵ or COR³⁵, C₁₋₇alkyl which may be partially unsaturated and optionally substituted by R³⁶, C₂₋₇alkyl which may be partially unsaturated and optionally substituted by R³³, or C₃₋₈cycloalkyl which may be partially unsaturated and optionally substituted by R³⁶, R³³ or R³⁴;

each R²³ and R²⁴ is independently H, halo, aryl, S(O)ₘR³⁰, COR³⁰, cyano, het, CF₃, OR²⁹, OR³¹, SR²⁹, SR³¹, NR²⁵R²⁶, CH(OR²⁹)R²⁷, CO₂R²⁹, CH(CO₂R²⁹)₂, NHCOR²⁷, or NHS(O)₂R²⁷ or C₁₋₇alkyl which may be partially unsaturated and optionally substituted by R²⁸;

each R²⁵ and R²⁶ is independently H or C₁₋₇alkyl;

R²⁷ is C₁₋₇alkyl optionally substituted by R³⁶ or C₂₋₇alkyl optionally substituted by R³³;

R²⁸ is cyano, halo, CF₃, aryl, het, C(═O)C₁₋₇ alkyl, CO₂C₁₋₇alkyl, OR²⁹, OR³¹, OR³², SR²⁹, SR³¹, SR³², NR²⁵R²⁶, CH(OR²⁹)R²⁷, CO₂R²⁹ or CH(CO₂R²⁹)₂;

R²⁹ is H or C₁₋₇alkyl;

R³⁰ is C₁₋₇alkyl, NR²⁵R²⁶, aryl or het;

R³¹ is C₂₋₇alkyl substituted by OH;

R³² is (P═)(OR²⁹)₂, CO(CH₂)ₙCON(CH₃)—(CH₂)ₙSO₃⁻M⁺, an amino acid, C(═O)aryl, or C(═O)C₁₋₇alkyl optionally substituted by NR²⁵R²⁶, aryl, het, carboxy, or O(CH₂)ₙCO₂R²⁹;

R³³ is hydroxy or NR²⁵R²⁶;

R³⁴ is C₁₋₇alkyl optionally substituted R³³;

R³⁵ is C₁₋₇alkyl, aryl or het;

R³⁶ is CO₂H or CO₂C₁₋₇alkyl

each n is independently 1, 2, 3, 4, or 5;

each m is independently 0, 1, or 2;

M is a pharmaceutically acceptable cation (e.g. sodium, potassium, or lithium);

wherein any aryl or het are the same as in Formula I, and are optionally substituted with one or more substituents (e.g. 1, 2, 3, 4, or 5) independently selected from the group consisting of halo, cyano, trifluoromethyl, trifluoromethoxy, hydroxy, carboxy, OR²⁷, phenyl, phenoxy, (C₁₋₇alkoxy)carbonyl, SR³¹, and C₁₋₇ alkyl optionally substituted with one or more substituents independently selected from the group consisting of cyano, aryl, mercapto, het, R³⁶, OR³⁷, SR³⁷, and SR³¹; wherein phenyl or phenoxy is optionally substituted with one or more substituents independently selected from cyano, halo, trifluoromethyl, trifluoromethoxy, carboxy, het, OR³¹, and R²⁷ ;

a preferred method of using compounds of Formula I′ as described above, uses those compounds, with the proviso that when R²¹ is Cl, and R²² is H, R²³ is other than CH₂OH;

wherein Formula II is

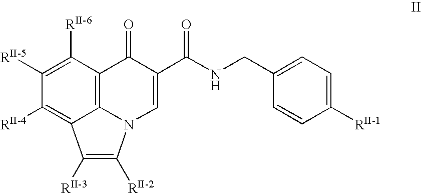

or a pharmaceutically acceptable salt, racemate, solvate, tautomer, optical isomer or prodrug derivative thereof wherein:

R^{II-1} is

(a) F,

(b) Cl,

(c) Br,

(d) CN or

(e) NO₂;

R^{II-2} and R^{II-3} are independently

(a) H,

(b) halo,

(c) OR^{II-1},

(d) C(═O)R^{II-7},

(e) C(═O)OR^{II-11},

(f) C₃₋₈cycloalkyl, or

(g) C₁₋₇alkyl which may be partially unsaturated and optionally substituted by one or more halo, C₃₋₈cycloalkyl, R^{II-12}, OR^{II-14}, SR^{II-11}, SR^{II-14}, NR^{II-8}, R^{II-9}, NR^{II-11}, C(O)R^{II-7}, (C═O)C₁₋₇ alkyl, or SOₘR^{II-10};

R^{II-4}, and R^{II-5} are independently

(a) H,

(b) halo,

(c) aryl,

(d) S(O)ₘR^{II-7},

(e) (C═O)R^{II-7},

(f) (C═O)OR^{II-10},

(g) CN,

(h) het, wherein said het is bound via a carbon atom,

(i) OR^{II-11},

(j) Ohet,

(k) NR^{II-8}R^{II-9}

(l) SR^{II-11},

(m) Shet,

(n) NHCOR^{II-13},

(o) NHSO₂R^{II-13},

(p) C₃₋₈cycloalkyl, or

(q) C₁₋₇alkyl which may be partially unsaturated and optionally substituted by one or more R^{II-12}, OR^{II-14}, SR^{II-11}, SR^{II-14}, NR^{II-8}R^{II-9}, halo, C₃₋₈cycloalkyl, (C═O)C₁₋₇alkyl, or SOₘR^{II-10};

R^{II-6} is

(a) H,

(b) halo,

(c) C₃₋₈cycloalkyl, or

(d) C₁₋₄alkyl optionally substituted by 1-3 halo;

R^{II-7} is

(a) C₁₋₇alkyl,

(b) C₃₋₈cycloalkyl,

(c) NR^{II-8}R^{II-9},

(d) aryl, or

(e) het, wherein said het is bonded via a carbon atom;

R^{II-8} and R^{II-9} are independently

(a) H,

(b) aryl,

(c) C₃₋₈cycloalkyl,

(d) C₁₋₇alkyl which may be partially unsaturated and is optionally substituted by one or more NR^{II-11}R^{II-1}, R^{II-12}, SOₘR^{II-10}, CONR^{II-11}R^{II-11}, OH aryl, het, C₃₋₈cycloalkyl, or halo, or

(e) R^{II-8} and R^{II-9} together with the nitrogen to which they are attached for a het;

R^{II-10} is

(a) aryl,

(b) het,

(c) C₃₋₈cycloalkyl, or

(d) C₁₋₇alkyl which may be partially unsaturated and is optionally substituted by one or more NR^{II-11}R^{II-11}, R^{II-12}, SH, CONR^{II-11}R^{II-11}, C₃₋₈cycloalkyl, or halo;

R^{II-11} is

(a) H,

(b) aryl,

(c) C₃₋₈cycloalkyl, or

(d) C₁₋₇alkyl optionally substituted by OH;

R^{II-12} is

(a) OR^{II-11},

(b) Ohet,

(c) Oaryl,

(d) CO₂R^{II-11},

(e) het,

(f) aryl, or

(g) CN;

R^{II-13} is

(a) H,

(b) het,

(c) aryl,

(d) C₃₋₈cycloalkyl, or

(e) C₁₋₇alkyl optionally substituted by NR^{II-11}R^{II-11} or R^{II-12};

R^{II-14} is

(a) (P═O)(OR^{II-15})₂,

(b) CO(CH₂)ₙCON(CH₃)—(CH₂)ₙSO₃⁻M⁺,

(c) an amino acid,

(d) C(═O)aryl, or

(e) C(═O)C₁₋₇alkyl optionally substituted by NR^{II-11}R^{II-11}, aryl, het, CO₂H, or O(CH₂)ₙCO₂R^{II-15};

R^{II-15} is

(a) H, or

(b) C₁₋₇alkyl;

aryl, in Formula II, is a phenyl radical or an ortho-fused bicyclic carbocyclic radical wherein at least one ring is aromatic; at each occurrence, aryl may be additionally substituted with one or more halo, CN, CO₂R^{II-11}, SR^{II-11}, OR^{II-11}, NR^{II-11}R^{II-11}, C₁₋₄alkyl, CF₃, or C₃₋₈cycloalkyl;

het, in Formula II, is a four-(4), five-(5), six-(6), or seven-(7) membered saturated or unsaturated heterocyclic ring having 1, 2, or 3 heteroatoms selected from the group consisting of O, SOₘ, and NX; wherein X is H, C₁₋₄alkyl or absence, wherein het is optionally fused to a benzene ring, or any bicyclic heterocycle group; at each occurrence, het may be additionally substituted with one or more halo, CN, CO₂R^{II-11}, SR^{II-11}, OR^{II-11}, NR^{II-11}R^{II-11}, C(═O)R^{II-13}, C₁₋₄alkyl, CF₃, C₃₋₈cycloalkyl, oxo or oxime;

at each occurrence, m is independently 0, 1, or 2;

at each occurrence, n is independently 1, 2, 3, 4, 5 or 6; and

M is sodium, potassium, or lithium.

The advantage of using compounds of Formula I, I′ and II in the method of our invention is their extensive activity against herpesviruses since atherosclerosis is related to the number of herpesvirus infections. Drugs containing compounds of Formulae I, I′ and II could prevent the inflammatory response resulting from reactivation of HCMV, EBV, HSV-1, HSV-2, HHV-8 and VZV.

### DETAILED DESCRIPTION OF THE INVENTION

The compounds of Formula I and I′, their method of preparation and formulation into pharmaceutical dosage forms are described in U.S. Pat. No. 6,239,142. The disclosure is herein incorporated in its entirety by reference.

The compounds of Formula II, their method of preparation and formulation into pharmaceutical dosage form are described in U.S. application Ser. No. 09/888,283, filed Jun. 22, 2001. The disclosure is herein incorporated in its entirety by reference.

The correspondence between the compounds utilized in the method of the invention and the compounds incorporated by reference is as follows:

Formulae I and I′ corresponds to Formulas I and II, respectively, of U.S. Pat. No. 6,239,142.

Formula II corresponds to Formula I of U.S. application Ser. No. 09/888,283.

Also provided are the following methods of treating or preventing atherosclerosis or restenosis in a mammal comprising administering to said mammal an effective amount of a compound of Formulae I, I′ or II.

A method of treating atherosclerosis or restenosis in a mammal wherein the compound administered has the Formula I and wherein R¹ is F, Cl or Br.

A method of treating atherosclerosis or restenosis in an animal, wherein the compound administered has the Formula I and wherein R¹ is Cl.

A method of treating atherosclerosis or restenosis in an animal, wherein the compound administered has the Formula I and wherein R² is H.

A method of treating atherosclerosis or restenosis in an animal, wherein the compound administered has the Formula I and wherein R² is H.

A method of treating atherosclerosis or restenosis in an animal, wherein the compound administered has the Formula I and wherein R² is H.

A method of treating atherosclerosis or restenosis in an animal, wherein the compound administered has the Formula I and wherein R² is R⁵, NR⁷R⁸, SO₂R⁹, or OR⁹.

A method of treating atherosclerosis or restenosis in an animal, wherein the compound administered has the Formula I and wherein R² is R⁵, NR⁷R⁸, SO₂R⁹, or OR⁹.

A method of treating atherosclerosis or restenosis in an animal, wherein the compound administered has the Formula I and wherein R² is R⁵, NR⁷P⁸, SO₂R⁹, or OR⁹.

A method of treating atherosclerosis or restenosis in an animal, wherein the compound administered has the Formula I and wherein R² is R⁵and R⁵ is C₁₋₇alkyl which may be partially unsaturated and is optionally substituted by one or more substituents selected from a group consisting of NR⁷R⁸, R¹¹, SOₘR⁹, and OC₂₋₄alkyl, which may be further substituted by het, OR¹⁰, or NR⁷R⁸.

A method of treating atherosclerosis or restenosis in an animal, wherein the compound administered has the Formula I and wherein R² is R⁵ and R⁵ is C₁₋₇alkyl which may be partially unsaturated and is optionally substituted by one or more substituents selected from a group consisting of NR⁷R⁸, R¹¹, SOₘR⁹, and OC₂₋₄alkyl, which may be further substituted by het, OR¹⁰, or NR⁷R⁸.

A method of treating atherosclerosis or restenosis in an animal, wherein the compound administered has the Formula I and wherein R² is R⁵ and R⁵ is C₁₋₇alkyl which may be partially unsaturated and is optionally substituted by one or more substituents selected from a group consisting of NR⁷ R⁸, R¹¹, SOₘR⁹, and OC₂₋₄alkyl, which may be further substituted by het, OR¹⁰, or NR⁷R⁸.

A method of treating atherosclerosis or restenosis in an animal, wherein the compound administered has the Formula I and wherein R² is R⁵ and R⁵ is C₁₋₇alkyl, which may be partially unsaturated and is optionally substituted by one or more aryl or het.

A method of treating atherosclerosis or restenosis in an animal, wherein the compound administered has the Formula I and wherein R² is R⁵ and R⁵ is C₁₋₇alkyl, which may be partially unsaturated and is optionally substituted by one or more aryl or het.

A method of treating atherosclerosis or restenosis in an animal, wherein the compound administered has the Formula I and wherein R² is R⁵ and R⁵ is C₁₋₇alkyl, which may be partially unsaturated and is optionally substituted by one or more aryl or het.

A method of treating atherosclerosis or restenosis in an animal, wherein the compound administered has the Formula I and wherein R⁵ is C₁₋₇alkyl.

A method of treating atherosclerosis or restenosis in an animal, wherein the compound administered has the Formula I and wherein R² is methyl, ethyl, propyl, isopropyl, butyl, tert-butyl, carboxymethyl, (C₁₋₇ alkoxy)carbonylmethyl, 2-hydroxyethyl, 2-(2-methoxy-ethoxy)ethyl, 3-(2-tetrahydropyranyloxy)propyl, 2-morpholinoethyl, 2-(diethylamino)ethyl, 2-(dimethyl-amino)ethyl, 2-piperidinoethyl, 3-piperidinopropyl, 2-(1-methylpyrrolidin-2-yl)ethyl, 2-(diisopropylamino)ethyl, 2-pyrrolidin-1-ylethyl, 3-(dimethylamino)propyl, benzyl, 3-fluorobenzyl, 3-phenylpropyl, 2-tetrahydrofuranylmethyl, 2-pyrrolidinoethyl, 3-pyridylmethyl, or vinyl.

A method of treating atherosclerosis or restenosis in an animal, wherein the compound administered has the Formula I and wherein R² is methyl, ethyl, isopropyl, 2-hydroxyethyl, 2-(diethylamino)ethyl, or 2-(dimethylamino)ethyl.

A method of treating atherosclerosis or restenosis in an animal, wherein the compound administered has the Formula I and wherein R³ is H, halo, S(O)ₘR⁶, (C═O)R⁶, (C═O)OR⁹, cyano, or C₁₋₇alkyl, which may be partially unsaturated and optionally substituted by one or more substituents of the group R¹¹, OR¹³, SR¹⁰, SR¹³, NR⁷R⁸, halo, (C═O)C₁₋₇alkyl, and SOₘR⁹.

A method of treating atherosclerosis or restenosis in an animal, wherein the compound administered has the Formula I and wherein R³ is C₁₋₇alkyl which may be partially unsaturated and optionally substituted by one or more substituents of the group R¹¹, OR¹³, SR¹⁰, SR¹³, NR⁷R⁸, halo, (C═O)C₁₋₇alkyl, and SOₘR⁹.

A method of treating atherosclerosis or restenosis in an animal, wherein the compound administered has the Formula I and wherein R³ is C₁₋₇alkyl which may be partially unsaturated and is substituted by one or more substituents of the group R¹¹, OR¹³, SR¹⁰, SR¹³, NR⁷R⁸, halo, (C═O)C₁₋₇alkyl, and SOₘR⁹.

A method of treating atherosclerosis or restenosis in an animal, wherein the compound administered has the Formula I and wherein R³ is C₁₋₇alkyl which may be partially unsaturated and is substituted by one or more substituents of the group OR¹⁰, het and NR⁷R⁸.

A method of treating atherosclerosis or restenosis in an animal, wherein the compound administered has the Formula I and wherein R³ is bromo, iodo, 3-hydroxy-1-propynyl, 3-methoxy-1-propynyl, 4-hydroxy-1-butynyl, 3-hydroxypropyl, cyano, 4,4-di(methoxycarbonyl)-1-butynyl, 4-hydroxybutyl, 3-(3-carboxypropanoyloxy)-1-propynyl, 3-(morpholinoacetoxy)-1-propynyl, 3-(2-amino-3-methylbutanoyloxy)-1-propynyl, or thiomorpholinomethyl, N-[2-(4-hydroxyphenyl)-2-hydroxyethyl]-N-(methyl)aminomethyl, morpholinocarbonyl, 3-[3-(morpholinomethyl)benzoyloxy]-1-propynyl.

A method of treating atherosclerosis or restenosis in an animal, wherein the compound administered has the Formula I and wherein R³ is iodo, 3-hydroxy-1-propynyl, 4-hydroxy-1-butynyl, 3-hydroxypropyl, morpholimomethyl, N-[2-(4-hydroxyphenyl)-2-hydroxyethyl]-N-(methyl)aminomethyl or 4-hydroxybutyl.

A method of treating atherosclerosis or restenosis in an animal, wherein the compound administered has the Formula I and wherein R³ is 3-hydroxy-1-propynyl, morpholimomethyl, N-[2-(4-hydroxyphenyl)-2-hydroxyethyl]-N-(methyl)aminomethyl or 3-hydroxypropyl.

A method for treating atherosclerosis or restenosis in a mammal, wherein the compound administered has the Formula I, and is:

(1) N-(4-Chlorobenzyl)-4-hydroxythieno[2,3-b]pyridine-5-carboxamide;

(2) N-(4-Chlorobenzyl)-4-hydroxy-2-iodothieno[2,3-b]pyridine-5-carboxamide;

(3) N-(4-Chlorobenzyl)-4-hydroxy-2-(4-morpholinylsulfonyl)thieno[2,3-b]-pyridine-5-carboxamide;

(4) 2-Bromo-N-(4-chlorobenzyl)-4-hydroxythieno[2,3-b]pyridine-5-carboxamide;

(5) N-(4-Chlorobenzyl)-4-hydroxy-2-(3-hydroxy-1-propynyl)thieno[2,3-b]-pyridine-5-carboxamide;

(6) N-(4-Chlorobenzyl)-4-hydroxy-2-(3-methoxy-1-propynyl)thieno[2,3-b]-pyridine-5-carboxamide;

(7) N-(4-Chlorobenzyl)-4-hydroxy-2-(4-hydroxy-1-butynyl)thieno[2,3-b]-pyridine-5-carboxamide;

(8) N-(4-Chlorobenzyl)-4-hydroxy-2-(3-hydroxypropyl)thieno[2,3-b]pyridine-5-carboxamide;

(9) N-(4-Chlorobenzyl)-2-cyano-4-hydroxythieno[2,3-b]pyridine-5-carboxamide;

(10) Dimethyl 2-[3-(5-{[(4-chlorobenzyl)amino]carbonyl}-4-hydroxythieno[2,3-b]-pyridin-2-yl)-2-propynyl]malonate;

(11) 2-Bromo-N-(4-chlorobenzyl)-7-ethyl-4-oxo-4,7-dihydrothieno[2,3-b]-pyridine-5-carboxamide;

(12) N-(4-Chlorobenzyl)-7-ethyl-4-oxo-4,7-dihydrothieno[2,3-b]pyridine-5-carboxamide;

(13) N-(4-Chlorobenzyl)-7-ethyl-2-iodo-4-oxo-4,7-dihydrothieno[2,3-b]-pyridine-5-carboxamide;

(14) N-(4-Chlorobenzyl)-7-ethyl-2-(3-hydroxy-1-propynyl)-4-oxo-4,7-dihydrothieno[2,3-b]pyridine-5-carboxamide;

(15) N-(4-Chlorobenzyl)-7-ethyl-2-(4-hydroxy-1-butynyl)-4-oxo-4,7-dihydrothieno[2,3-b]pyridine-5-carboxamide;

(16) N-(4-Chlorobenzyl)-7-ethyl-2-(3-hydroxypropyl)-4-oxo-4,7-dihydrothieno[2,3-b]pyridine-5-carboxamide;

(17) N-(4-Chlorobenzyl)-7-(2-hydroxyethyl)-2-(3-hydroxy-1-propynyl)-4-oxo-4,7-dihydrothieno[2,3-b]pyridine-5-carboxamide;

(18) N-(4-Chlorobenzyl)-7-[2-(diethylamino)ethyl]-2-(3-hydroxy-1-propynyl)-4-oxo-4,7-dihydrothieno[2,3-b]pyridine-5-carboxamide;

(19) 2-[5-{[(4-Chlorobenzyl)amino]carbonyl}-2-(3-hydroxy-1-propynyl)-4-oxothieno[2,3-b]pyridin-7(4H)-yl]acetic acid;

(20) N-(4-Chlorobenzyl)-7-ethyl-2-(4-hydroxybutyl)-4-oxo-4,7-dihydrothieno[2,3-b]pyridine-5-carboxamide;

(21) N-(4-Chlorobenzyl)-7-(2-hydroxyethyl)-2-(3-hydroxypropyl)-4-oxo-4,7-dihydrothieno[2,3-b]pyridine-5-carboxamide;

(22) N-(4-Chlorobenzyl)-7-[2-(diethylamino)ethyl]-2-(3-hydroxypropyl)-4-oxo-4,7-dihydrothieno[2,3-b]pyridine-5-carboxamide;

(23) N-(4-Chlorobenzyl)-2-iodo-7-methyl-4-oxo-4,7-dihydrothieno[2,3-b]-pyridine-5-carboxamide;

(24) N-(4-Chlorobenzyl)-2-(3-hydroxy-1-propynyl)-7-methyl-4-oxo-4,7-dihydrothieno[2,3-b]pyridine-5-carboxamide;

(25) N-(4-Chlorobenzyl)-2-(3-hydroxypropyl)-7-methyl-4-oxo-4,7-dihydrothieno[2,3-b]pyridine-5-carboxamide;

(26) N-(4-Chlorobenzyl)-2-iodo-7-isopropyl-4-oxo-4,7-dihydrothieno[2,3-b]-pyridine-5-carboxamide;

(27) N-(4-Chlorobenzyl)-2-(3-hydroxy-1-propynyl)-7-isopropyl-4-oxo-4,7-dihydrothieno[2,3-b]pyridine-5-carboxamide;

(28) N-(4-Chlorobenzyl)-2-(3-hydroxypropyl)-7-isopropyl-4-oxo-4,7-dihydrothieno[2,3-b]pyridine-5-carboxamide;

(29) 4-{[3-(5-{[(4-Chlorobenzyl)amino]carbonyl}-7-ethyl-4-oxo-4,7-dihydrothieno[2,3-b]pyridin-2-yl)-2-propynyl]oxy}-4-oxobutanoic acid;

(30) 3-(5-{[(4-Chlorobenzyl)amino]carbonyl}-7-ethyl-4-oxo-4,7-dihydrothieno[2,3-b]pyridin-2-yl)-2-propynyl 2-(4-morpholinyl)acetate;

(31) 3-(5-{[(4-Chlorobenzyl)amino]carbonyl}-7-ethyl-4-oxo-4,7-dihydrothieno[2,3-b]pyridin-2-yl)-2-propynyl 2-amino-3-methylbutanoate;

(32) 3-(5-{[(4-Chlorobenzyl)amino]carbonyl}-7-ethyl-4-oxo-4,7-dihydrothieno[2,3-b]pyridin-2-yl)-2-propynyl 3-(4-morpholinylmethyl)benzoate;

(33) Methyl-5-{[4-chlorobenzyl)amino]carbonyl}-4-hydroxythienol[2,3-b]pyridine-2-carboxylate;

(34) N-(4-Chlorobenzyl)-4-hydroxy-2-(hydroxymethyl)thieno[2,3-b]pyridine-5-carboxamide;

(35) N-(4-chlorobenzyl)-2-(hydroxymethy)-7-methyl-4-oxo-4,7-dihydrothienol[2,3-b]pyridine-5-carboxamide;

(36) N-(4-chlorobenzyl)-7-methyl-2-(4-morpholinylmethyl)-4-oxo-4,7-dihydrothienol[2,3-b]pyridine-5-carboxamide;

(37) N-(4-chlorobenzyl)-7-methyl-2-(4-morpholinylmethyl)-4-oxo-4,7-dihydrothieno[2,3-b]pyridine-5-carboxamide;

(38) N-(4-chlorobenzyl)-7-methyl-4-oxo-2-(4-thiomorpholinylmethyl)-4,7-dihydrothieno[2,3-b]pyridine-5-carboxamide;

(39) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b]pyridine-5-carboxamide;

(40) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;

(41) N-(4-chlorobenzyl)-4-hydroxy-2-(4-morpholinylmethyl)thieno[2,3-b]pyridine-5-carboxamide;

(42) N-(4-Chlorobenzyl)-7-ethyl-2-(4-morpholinylmethyl)-4-oxo-4,7-dihydrothieno[2,3-b]pyridine-5-carboxamide;

(43) N-(4-Chlorobenzyl)-2-(4-morpholinylmethyl)-4-oxo-7-propyl-4,7-dihydrothieno[2,3-b]pyridine-5-carboxamide;

(44) N-(4-Chlorobenzyl)-7-isopropyl-2-(4-morpholinylmethyl)-4-oxo-4,7-dihydrothieno[2,3-b]pyridine-5-carboxamide;

(45) N-(4-Fluorobenzyl)-7-methyl-2-(4-morpholinylmethyl)-4-oxo-4,7-dihydrothieno[2,3-b]pyridine-5-carboxamide;

(46) N-(4-bromobenzyl)-7-methyl-2-(4-morpholinylmethyl)-4-oxo-4,7-dihydrothieno(2,3-b]pyridine-5-carboxamide;

(47) N-(4-chlorobenzyl)-4-hydroxy-2-(4-morpholinylcarbonyl)thieno[2,3-b]pyridine-5-carboxamide;

(48) N-(4-chlorobenzyl)-7-methyl-2-(4-morpholinylcarbonyl)-4-oxo-4,7-dihydrothieno[2,3-b]pyridine-5-carboxamide;

(49) 7-Benzyl-N-(4-chlorobenzyl)-2-(4-morpholinylmethyl)-4-oxo-4,7-dihydrothieno[2,3-b]pyridine-5-carboxamide;

(50) N-(4-Chlorobenzyl)-7-(3-fluorobenzyl)-2-(4-morpholinylmethyl)-4-oxo-4,7-dihydrothieno[2,3-b]pyridine-5-carboxamide;

(51) N-(4-Chlorobenzyl)-2-(4-morpholinylmethyl)-4-oxo-7-(3-phenylpropyl)-4,7-dihydrothieno[2,3-b]pyridine-5-carboxamide;

(52) N-(4-Chlorobenzyl)-2-(4-morpholinylmethyl)-4-oxo-7-(tetrahydro-2-furanylmethyl)-4,7-dihydrothieno[2,3-b]pyridine-5-carboxamide;

(53) N-(4-Chlorobenzyl)-2-(4-morpholinylmethyl)-4-oxo-7-[2-(1-pyrrolidinyl)ethyl]-4,7-dihydrothieno[2,3-b]pyridine-5-carboxamide;

(54) N-(4-Chlorobenzyl)-2-(4-morpholinylmethyl)-4-oxo-7-(3-pyridinylmethyl)-4,7-dihydrothieno[2,3-b]pyridine-5-carboxamide;

(55) N-(4-Chlorobenzyl)-2-(4-morpholinylmethyl)-4-oxo-7-(4-pyridinylmethyl)-4,7-dihydrothieno[2,3-b]pyridine-5-carboxamide; or

a pharmaceutically acceptable salt thereof.

A method for treating atherosclerosis or restenosis in a mammal, wherein the compound administered has the Formula I, and is:

(1) N-(4-Chlorobenzyl)-7-ethyl-2-(3-hydroxy-1-propynyl)-4-oxo-4,7-dihydro-thieno[2,3-b]pyridine-5-carboxamide;

(2) N-(4-Chlorobenzyl)-7-ethyl-2-(4-hydroxy-1-butynyl)-4-oxo-4,7-dihydro-thieno[2,3-b]pyridine-5-carboxamide;

(3) N-(4-Chlorobenzyl)-7-ethyl-2-(3-hydroxypropyl)-4-oxo-4,7-dihydro-thieno[2,3-b]pyridine-5-carboxamide;

(4) N-(4-Chlorobenzyl)-7-(2-hydroxyethyl)-2-(3-hydroxy-1-propynyl)-4-oxo-4,7-dihydrothieno[2,3-b]pyridine-5-carboxamide;

(5) N-(4-Chlorobenzyl)-7-[2-(diethylamino)ethyl]-2-(3-hydroxy-1-propynyl)-4-oxo-4,7-dihydrothieno[2,3-b]pyridine-5-carboxamide;

(6) N-(4-Chlorobenzyl)-7-ethyl-2-(4-hydroxybutyl)-4-oxo-4,7-dihydro-thieno[2,3-b]pyridine-5-carboxamide;

(7) N-(4-Chlorobenzyl)-7-(2-hydroxyethyl)-2-(3-hydroxypropyl)-4-oxo-4,7-dihydrothieno[2,3-b]pyridine-5-carboxamide;

(8) N-(4-Chlorobenzyl)-7-[2-(diethylamino)ethyl]-2-(3-hydroxypropyl)-4-oxo-4,7-dihydrothieno[2,3-b]pyridine-5-carboxamide;

(9) N-(4-Chlorobenzyl)-2-iodo-7-methyl-4-oxo-4,7-dihydrothieno[2,3-b]-pyridine-5-carboxamide;

(10) N-(4-Chlorobenzyl)-2-(3-hydroxy-1-propynyl)-7-methyl-4-oxo-4,7-dihydrothieno[2,3-b]pyridine-5-carboxamide;

(11) N-(4-Chlorobenzyl)-2-(3-hydroxypropyl)-7-methyl-4-oxo-4,7-dihydro-thieno[2,3-b]pyridine-5-carboxamide;

(12) N-(4-Chlorobenzyl)-2-iodo-7-isopropyl-4-oxo-4,7-dihydrothieno[2,3-b]-pyridine-5-carboxamide;

(13) N-(4-Chlorobenzyl)-2-(3-hydroxy-1-propynyl)-7-isopropyl-4-oxo-4,7-dihydrothieno[2,3-b]pyridine-5-carboxamide;

(14) N-(4-Chlorobenzyl)-2-(3-hydroxypropyl)-7-isopropyl-4-oxo-4,7-dihydro-thieno[2,3-b]pyridine-5-carboxamide;

(15) 4-{[3-(5-{[(4-Chlorobenzyl)amino]carbonyl}-7-ethyl-4-oxo-4,7-dihydro-thieno[2,3-b]pyridin-2-yl)-2-propynyl]oxy}-4-oxobutanoic acid;

(16) 3-(5-{[(4-Chlorobenzyl)amino]carbonyl}-7-ethyl-4-oxo-4,7-dihydro-thieno[2,3-b]pyridin-2-yl)-2-propynyl 2-(4-morpholinyl)acetate;

(17) 3-(5-{[(4-Chlorobenzyl)amino]carbonyl}-7-ethyl-4-oxo-4,7-dihydro-thieno[2,3-b]pyridin-2-yl)-2-propynyl 2-amino-3-methylbutanoate;

(18) 3-(5-{[(4-Chlorobenzyl)amino]carbonyl}-7-ethyl-4-oxo-4,7-dihydro-thieno[2,3-b]pyridin-2-yl)-2-propynyl 3-(4-morpholinylmethyl)benzoate;

(19) N-(4-chlorobenzyl)-7-methyl-2-(4-morpholinylmethyl)-4-oxo-4,7-dihydrothienol[2,3-b]pyridine-5-carboxamide;

(20) N-(4-chlorobenzyl)-7-methyl-2-(4-morpholinylmethyl)-4-oxo-4,7-dihydrothieno[2,3-b]pyridine-5-carboxamide;

(21) N-(4-chlorobenzyl)-7-methyl-4-oxo-2-(4-thiomorpholinylmethyl)-4,7-dihydrothieno[2,3-b]pyridine-5-carboxamide;

(22) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b]pyridine-5-carboxamide;

(23) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;

(24) N-(4-Chlorobenzyl)-7-ethyl-2-(4-morpholinylmethyl)-4-oxo-4,7-dihydrothieno[2,3-b]pyridine-5-carboxamide;

(25) N-(4-Chlorobenzyl)-2-(4-morpholinylmethyl)-4-oxo-7-propyl-4,7-dihydrothieno[2,3-b]pyridine-5-carboxamide;

(26) N-(4-Chlorobenzyl)-7-isopropyl-2-(4-morpholinylmethyl)-4-oxo-4,7-dihydrothieno[2,3-b]pyridine-5-carboxamide;

(27) N-(4-Fluorobenzyl)-7-methyl-2-(4-morpholinylmethyl)-4-oxo-4,7-dihydrothieno[2,3-b]pyridine-5-carboxamide;

(28) N-(4-bromobenzyl)-7-methyl-2-(4-morpholinylmethyl)-4-oxo-4,7-dihydrothieno[2,3-b]pyridine-5-carboxamide;

(29) 7-Benzyl-N-(4-chlorobenzyl)-2-(4-morpholinylmethyl)-4-oxo-4,7-dihydrothieno[2,3-b]pyridine-5-carboxamide;

(30) N-(4-Chlorobenzyl)-7-(3-fluorobenzyl)-2-(4-morpholinylmethyl)-4-oxo-4,7-dihydrothieno[2,3-b]pyridine-5-carboxamide;

(31) N-(4-Chlorobenzyl)-2-(4-morpholinylmethyl)-4-oxo-7-(3-phenylpropyl)-4,7-dihydrothieno[2,3-b]pyridine-5-carboxamide;

(32) N-(4-Chlorobenzyl)-2-(4-morpholinylmethyl)-4-oxo-7-(tetrahydro-2-furanylmethyl)-4,7-dihydrothieno[2,3-b]pyridine-5-carboxamide;

(33) N-(4-Chlorobenzyl)-2-(4-morpholinylmethyl)-4-oxo-7-[2-(1-pyrrolidinyl)ethyl]-4,7-dihydrothieno[2,3-b]pyridine-5-carboxamide;

(34) N-(4-Chlorobenzyl)-2-(4-morpholinylmethyl)-4-oxo-7-(3-pyridinylmethyl)-4,7-dihydrothieno[2,3-b]pyridine-5-carboxamide;

(35) N-(4-Chlorobenzyl)-2-(4-morpholinylmethyl)-4-oxo-7-(4-pyridinylmethyl)-4,7-dihydrothieno[2,3-b]pyridine-5-carboxamide; or

a pharmaceutically acceptable salt thereof.

A method for treating atherosclerosis or restenosis in a mammal, wherein the compound administered has the Formula I, and is:

(1) N-(4-Chlorobenzyl)-7-ethyl-2-(3-hydroxy-1-propynyl)-4-oxo-4,7-dihydro-thieno[2,3-b]pyridine-5-carboxamide;

(2) N-(4-Chlorobenzyl)-7-ethyl-2-(4-hydroxy-1-butynyl)-4-oxo-4,7-dihydro-thieno[2,3-b]pyridine-5-carboxamide;

(3) N-(4-Chlorobenzyl)-7-ethyl-2-(3-hydroxypropyl)-4-oxo-4,7-dihydro-thieno[2,3-b]pyridine-5-carboxamide;

(4) N-(4-Chlorobenzyl)-7-(2-hydroxyethyl)-2-(3-hydroxy-1-propynyl)-4-oxo-4,7-dihydrothieno[2,3-b]pyridine-5-carboxamide;

(5) N-(4-Chlorobenzyl)-7-[2-(diethylamino)ethyl]-2-(3-hydroxy-1-propynyl)-4-oxo-4,7-dihydrothieno[2,3-b]pyridine-5-carboxamide;

(6) N-(4-Chlorobenzyl)-7-ethyl-2-(4-hydroxybutyl)-4-oxo-4,7-dihydro-thieno[2,3-b]pyridine-5-carboxamide;

(7) N-(4-Chlorobenzyl)-7-(2-hydroxyethyl)-2-(3-hydroxypropyl)-4-oxo-4,7-dihydrothieno[2,3-b]pyridine-5-carboxamide;

(8) N-(4-Chlorobenzyl)-7-[2-(diethylamino)ethyl]-2-(3-hydroxypropyl)-4-oxo-4,7-dihydrothieno[2,3-b]pyridine-5-carboxamide;

(9) N-(4-Chlorobenzyl)-2-iodo-7-methyl-4-oxo-4,7-dihydrothieno[2,3-b]-pyridine-5-carboxamide;

(10) N-(4-Chlorobenzyl)-2-(3-hydroxy-1-propynyl)-7-methyl-4-oxo-4,7-dihydrothieno[2,3-b]pyridine-5-carboxamide;

(11) N-(4-Chlorobenzyl)-2-(3-hydroxypropyl)-7-methyl-4-oxo-4,7-dihydro-thieno[2,3-b]pyridine-5-carboxamide;

(12) N-(4-Chlorobenzyl)-2-iodo-7-isopropyl-4-oxo-4,7-dihydrothieno[2,3-b]-pyridine-5-carboxamide;

(13) N-(4-Chlorobenzyl)-2-(3-hydroxy-1-propynyl)-7-isopropyl-4-oxo-4,7-dihydrothieno[2,3-b]pyridine-5-carboxamide;

(14) N-(4-Chlorobenzyl)-2-(3-hydroxypropyl)-7-isopropyl-4-oxo-4,7-dihydro-thieno[2,3-b]pyridine-5-carboxamide;

(15) N-(4-chlorobenzyl)-7-methyl-2-(4-morpholinylmethyl)-4-oxo-4,7-dihydrothieno[2,3-b]pyridine-5-carboxamide;

(16) N-(4-chlorobenzyl)-7-methyl-2-(4-morpholinylmethyl)-4-oxo-4,7-dihydrothieno[2,3-b]pyridine-5-carboxamide;

(17) N-(4-chlorobenzyl)-7-methyl-4-oxo-2-(4-thiomorpholinylmethyl)-4,7-dihydrothieno[2,3-b]pyridine-5-carboxamide;

(18) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b]pyridine-5-carboxamide;

(19) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;

(20) N-(4-Chlorobenzyl)-7-ethyl-2-(4-morpholinylmethyl)-4-oxo-4,7-dihydrothieno[2,3-b]pyridine-5-carboxamide;

(21) N-(4-Chlorobenzyl)-2-(4-morpholinylmethyl)-4-oxo-7-propyl-4,7-dihydrothieno[2,3-b]pyridine-5-carboxamide;

(22) N-(4-Chlorobenzyl)-7-isopropyl-2-(4-morpholinylmethyl)-4-oxo-4,7-dihydrothieno[2,3-b]pyridine-5-carboxamide;

(23) N-(4-Fluorobenzyl)-7-methyl-2-(4-morpholinylmethyl)-4-oxo-4,7-dihydrothieno[2,3-b]pyridine-5-carboxamide;

(24) N-(4-bromobenzyl)-7-methyl-2-(4-morpholinylmethyl)-4-oxo-4,7-dihydrothieno[2,3-b]pyridine-5-carboxamide;

(25) 7-Benzyl-N-(4-chlorobenzyl)-2-(4-morpholinylmethyl)-4-oxo-4,7-dihydrothieno[2,3-b]pyridine-5-carboxamide;

(26) N-(4-Chlorobenzyl)-7-(3-fluorobenzyl)-2-(4-morpholinylmethyl)-4-oxo-4,7-dihydrothieno[2,3-b]pyridine-5-carboxamide;

(27) N-(4-Chlorobenzyl)-2-(4-morpholinylmethyl)-4-oxo-7-(tetrahydro-2-furanylmethyl)-4,7-dihydrothieno[2,3-b]pyridine-5-carboxamide;

(28) N-(4-Chlorobenzyl)-2-(4-morpholinylmethyl)-4-oxo-7-[2-(1-pyrrolidinyl)ethyl]-4,7-dihydrothieno[2,3-b]pyridine-5-carboxamide;

(29) N-(4-Chlorobenzyl)-2-(4-morpholinylmethyl)-4-oxo-7-(3-pyridinylmethyl)-4,7-dihydrothieno[2,3-b]pyridine-5-carboxamide;

(30) N-(4-Chlorobenzyl)-2-(4-morpholinylmethyl)-4-oxo-7-(4-pyridinylmethyl)-4,7-dihydrothieno[2,3-b]pyridine-5-carboxamide; or

a pharmaceutically acceptable salt thereof.

A method for treating atherosclerosis or restenosis in a mammal, wherein the compound administered has the Formula I, and is:

(1) N-(4-Chlorobenzyl)-7-[2-(diethylamino)ethyl]-2-(3-hydroxypropyl)-4-oxo-4,7-dihydrothieno[2,3-b]pyridine-5-carboxamide;

(2) N-(4-Chlorobenzyl)-2-(3-hydroxy-1-propynyl)-7-methyl-4-oxo-4,7-dihydro-thieno[2,3-b]-pyridine-5-carboxamide;

(3) N-(4-Chlorobenzyl)-2-(3-hydroxypropyl)-7-methyl-4-oxo-4,7-dihydro-thieno[2,3-b]pyridine-5-carboxamide;

(4) N-(4-chlorobenzyl)-7-methyl-2-(4-morpholinylmethyl)-4-oxo-4,7-dihydrothieno[2,3-b]pyridine-5-carboxamide;

(5) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b]pyridine-5-carboxamide;

(6) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;

(7) N-(4-Chlorobenzyl)-7-ethyl-2-(4-morpholinylmethyl)-4-oxo-4,7-dihydrothieno[2,3-b]pyridine-5-carboxamide;

(8) N-(4-Chlorobenzyl)-2-(4-morpholinylmethyl)-4-oxo-7-propyl-4,7-dihydrothieno[2,3-b]pyridine-5-carboxamide; or

a pharmaceutically acceptable salt thereof.

A method for treating atherosclerosis or restenosis in a mammal, wherein the compound administered has the Formula I, and is N(4-chlorobenzyl)-7-methyl-2-(4-morpholinylmethyl)-4-oxo-4,7-dihydrothieno[2,3-b]pyridine-5-carboxamide or a pharmaceutically acceptable salt thereof.

A method of treating or preventing atherosclerosis in a mammal, wherein the compound administered has the Formula I′, and wherein a specific value for R²¹ is Cl.

A method of treating or preventing atherosclerosis in a mammal, wherein the compound administered has the Formula I′, and wherein a specific value for R²¹ is CN, or NO₂.

A method of treating or preventing atherosclerosis in a mammal, wherein the compound administered has the Formula I′, and wherein a specific value for R²² is H, —(CH₂CH₂O)ₙH, —(CH₂CH₂O)ₙCH₃, SO₂R³⁵, COR³⁵, C₁₋₇alkyl which may be partially unsaturated and optionally substituted by R³⁶, C₂₋₇alkyl which may be partially unsaturated and is optionally substituted by R³³, or C₃₋₈cycloalkyl which may be partially unsaturated and optionally substituted by R³⁶, R³³ or R³⁴.

A method of treating or preventing atherosclerosis in a mammal, wherein the compound administered has the Formula I′, and wherein a specific value for R²² is C₁₋₇alkyl which may be partially unsaturated and optionally substituted by R³⁶, C₂₋₇alkyl which may be partially unsaturated and is substituted by R³³, or C₃₋₈cycloalkyl which may be partially unsaturated and optionally substituted by R³⁶, R³³ or R³⁴.

A method of treating or preventing atherosclerosis in a mammal, wherein the compound administered has the Formula I′, and wherein a specific value for R²² is C₁₋₇ alkyl which may be substituted by R³⁶.

A method of treating or preventing atherosclerosis in a mammal, wherein the compound administered has the Formula I′, and wherein a specific value for R²² is C₂₋₇ alkyl which is partially unsaturated and is substituted by R³³.

A method of treating or preventing atherosclerosis in a mammal, wherein the compound administered has the Formula I′, and wherein a specific value for R²³ is independently H, halo, aryl, S(O)ₘR³⁰, COR³⁰, cyano, het, CF₃, OR²⁹, OR³¹, SR²⁹, SR³¹, NR²⁵R²⁶, CH(OR²⁹)R²⁷, CO₂R²⁹, CH(COOR²⁹)₂, NHCOR²⁷, or NHS(O)₂R²⁷ or C₁₋₇alkyl which may be partially unsaturated and optionally substituted by R²⁸.

A method of treating or preventing atherosclerosis in a mammal, wherein the compound administered has the Formula I′, and wherein a specific value for R²³ is independently halo, S(O)ₘR³⁰, COR³⁰, cyano, het, or C₁₋₇alkyl which may be partially unsaturated and optionally substituted by R²⁸ with the proviso that at least one of R²³ and R²⁴ is hydrogen.

A method of treating or preventing atherosclerosis in a mammal, wherein the compound administered has the Formula I′, and wherein a specific value for R²³ is independently C₁₋₇alkyl optionally substituted by R²⁸ with the proviso that at least one of R²³ and R²⁴ is hydrogen.

A method of treating or preventing atherosclerosis in a mammal, wherein the compound administered has the Formula I′, and wherein a specific value for R²³ is independently partially unsaturated C₁₋₇alkyl and optionally substituted by R²⁸ with the proviso that at least one of R²³ and R²⁴ is hydrogen.

A method of treating or preventing atherosclerosis in a mammal, wherein the compound administered has the Formula I′, and wherein a specific value for R²³ is independently (Z or E) —CH═CH(CH₂)ₙR²⁸ or —C≡C(CH₂)ₙR²⁸ with the proviso that at least one of R²³ and R²⁴ is hydrogen.

A method of treating or preventing atherosclerosis in a mammal, wherein the compound administered has the Formula I′, and wherein a specific value for R²² is methyl, ethyl, propyl, isopropyl, butyl, tert-butyl, carboxymethyl, (C₁₋₇ alkoxy)carbonylmethyl, 2-hydroxyethyl, 2-(2-methoxyethoxy)ethyl, 3-(2-tetrahydropyranyloxy)propyl, 2-morpholinoethyl, 2-(diethylamino)ethyl, 2-(dimethylamino)ethyl, 2-piperidinoethyl, 3-piperidinopropyl, 2-(1-methylpyrrolidin-2-yl)ethyl, 2-(diisopropylamino)ethyl, 2-pyrrolidin-1-ylethyl, 3-(dimethylamino)propyl, or vinyl.

A method of treating or preventing atherosclerosis in a mammal, wherein the compound administered has the Formula I′, and wherein a specific value for another more specific value for R²² is methyl, ethyl, isopropyl, 2-hydroxyethyl, 2-(diethylamino)ethyl, or 2-(dimethylamino)ethyl.

A method of treating or preventing atherosclerosis in a mammal, wherein the compound administered has the Formula I′, and wherein a specific value for another more specific value R²² is methyl, or 2-(dimethylamino)ethyl.

A method of treating or preventing atherosclerosis in a mammal, wherein the compound administered has the Formula I′, and wherein a specific value for another more specific value for R²³ is independently 3-hydroxy-1-propynyl, or 3-hydroxypropyl when R²⁴ is hydrogen.

A method of treating or preventing atherosclerosis in a mammal, wherein the compound administered has the Formula I′, and wherein R²¹ is Cl; R²² is —(CH₂CH₂O)ₙH, —(CH₂CH₂O)ₙCH₃, SO₂R³⁵, COR³⁵, C₁₋₇alkyl which may be partially unsaturated and optionally substituted by R³⁶, C₂₋₇alkyl which may be partially unsaturated and is substituted by R³³, or C₃₋₈cycloalkyl which may be partially unsaturated and optionally substituted by R³⁶, R³³ or R³⁴; each of R²³ and R²⁴ is independently C₁₋₇alkyl which may be partially unsaturated and optionally substituted by R²⁸ with the proviso that at least one of R²³ and R²⁴ is hydrogen; and R²⁸ is cyano, C₁₋₇ alkanoyl, OR²⁹, NR²⁵R²⁶, OR³¹, OR³², pyrrolidino, piperidino, morpholino, thiomorpholino; (CH₂)ₙOR²⁹, (CH₂)ₙOR³², (CH₂)ₙSR²⁹, CH(OR²⁹)R²⁷, (CH₂)ₙCN, C₁₋₇alkylCO₂R²⁹, or CH(COOR²⁹)₂; or a pharmaceutically acceptable salt thereof.

A method of treating or preventing atherosclerosis in a mammal, wherein the compound administered has the Formula I or I′ and wherein any aryl, or het is optionally substituted with one or two substituents selected from the group consisting of halo, cyano, het trifluoromethyl, trifluoromethoxy, hydroxy C₁₋₇alkoxy, and C₁₋₇alkyl; or a pharmaceutically acceptable salt thereof.

A method for treating or preventing atherosclerosis in a mammal, wherein the compound administered has the Formula II, and wherein R^{II-1} is Cl.

A method for treating or preventing atherosclerosis in a mammal, wherein the compound administered has the Formula II, and wherein R^{II-4} and R^{II-6} are hydrogen.

A method for treating or preventing atherosclerosis in a mammal, wherein the compound administered has the Formula II, and wherein R^{II-5} is C₁₋₇alkyl which may be partially unsaturated and optionally substituted by OH.

A method for treating or preventing atherosclerosis in a mammal, wherein the compound administered has the Formula II, and wherein R^{II-5} is C₁₋₇alkyl substituted by het.

A method for treating or preventing atherosclerosis in a mammal, wherein the compound administered has the Formula II, and wherein het is piperidinyl, morpholinyl, thiomorpholinyl, piperazinyl, pyrrolidinyl, pyridyl, imidazolyl, or tetrahydro-2H-pyran.

A method for treating or preventing atherosclerosis in a mammal, wherein the compound administered has the Formula II, and wherein het is morpholinyl.

A method for treating or preventing atherosclerosis in a mammal, wherein the compound administered has the Formula II, and wherein het is tetrahydro-2H-pyran.

A method for treating or preventing atherosclerosis in a mammal, wherein the compound administered has the Formula II, and wherein R^{II-5} is C₁₋₇alkyl substituted by NR^{II-8}R^{II-9}.

A method for treating or preventing atherosclerosis in a mammal, wherein the compound administered has the Formula II, and wherein R^{II-8} and R^{II-9} are independently H, or C₁₋₆alkyl optionally substituted by one to three OH, SH, halo, phenyl, or het.

A method for treating or preventing atherosclerosis in a mammal, wherein the compound administered has the Formula II, and wherein R^{II-8} and R^{II-9} are independently H, or C₁₋₆alkyl optionally substituted by one to two OH, or phenyl.

A method for treating or preventing atherosclerosis in a mammal, wherein the compound administered has the Formula II, and wherein R^{II-2} and R^{II-3} are independently hydrogen, or C₁₋₇alkyl which may be partially unsaturated and optionally substituted by OH.

A method for treating or preventing atherosclerosis in a mammal, wherein the compound administered has the Formula II, and wherein R^{II-3} is hydrogen.

A method for treating or preventing atherosclerosis in a mammal, wherein the compound administered has the Formula II, and wherein R^{II-3} is halo.

A method for treating or preventing atherosclerosis in a mammal, wherein the compound administered has the Formula II, and wherein R^{II-2} is hydroxymethyl.

A method for treating or preventing atherosclerosis in a mammal, wherein the compound administered has the Formula II, and wherein R^{II-2} is hydroxyethyl.

A method for treating or preventing atherosclerosis in a mammal, wherein the compound administered has the Formula II, and wherein R^{II-2} is C₁₋₇alkyl substituted by het.

A method for treating or preventing atherosclerosis in a mammal, wherein the compound administered has the Formula II, and wherein het is piperidinyl, morpholinyl, thiomorpholinyl, piperazinyl, pyrrolidinyl, pyridyl, imidazolyl, or tetrahydro-2H-pyran.

A method for treating or preventing atherosclerosis in a mammal, wherein the compound administered has the Formula II, and wherein het is 2-ethylpiperidinyl, 1,1-dioxido-4-thiomorpholinyl, 4-methylpiperazinyl.

A method for treating or preventing atherosclerosis in a mammal, wherein the compound administered has the Formula II, and wherein R^{II-2} is C₁₋₇alkyl substituted by NR^{II-8}R^{II-9}.

A method for treating or preventing atherosclerosis in a mammal, wherein the compound administered has the Formula II, and wherein R^{II-8} and R^{II-9} are independently H, or C₁₋₆alkyl optionally substituted by one to three OH, SH, CONR^{II-11}R^{II-11}, phenyl, or het.

A method for treating or preventing atherosclerosis in a mammal, wherein the compound administered has the Formula II, and wherein each is R^{II-11} is independently H, or C₁₋₆alkyl.

A method for treating or preventing atherosclerosis in a mammal, wherein the compound administered has the Formula II, and wherein R^{II-11} is H.

A method for treating or preventing atherosclerosis in a mammal, wherein the compound administered has the Formula II, and wherein R^{II-2} is C₁₋₇alkyl substituted by OR^{II-11} or SR^{II-11}.

A method for treating or preventing atherosclerosis in a mammal, wherein the compound administered has the Formula II, and wherein R^{II-1} is C₁₋₄ alkyl or phenyl.

A method for treating or preventing atherosclerosis in a mammal, wherein the compound administered has the Formula II, and wherein R^{II-1} is Cl; R₂ is hydrogen, or C₁₋₇alkyl which may be partially unsaturated and optionally substituted by OR^{II-11}, het, or NR^{II-8}R^{II-9}; R^{II-3} is hydrogen or halo; R^{II-4} and R^{II-6} are hydrogen; and R^{II-5} is C₁₋₇alkyl which may be partially unsaturated and optionally substituted by OH, het, or NR^{II-8}R^{II-9}.

A method of treating or preventing atherosclerosis or restenosis in a mammal, wherein the compound administered has the Formula II and is:

(a) N-(4-chlorobenzyl)-2-(hydroxymethyl)-6-oxo-6H-pyrrolo[3,2,1-ij]quinoline-5-carboxamide;

(b) N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide;

(c) N-(4-chlorobenzyl)-2-(hydroxymethyl)-8-(4-morpholinylmethyl)-6-oxo-6H-pyrrolo[3,2,1-ij]quinoline-5-carboxamide;

(d) N-(4-chlorobenzyl)-2-(2-hydroxyethyl)-8-(4-morpholinylmethyl)-6-oxo-6H-pyrrolo[3,2,1-ij]quinoline-5-carboxamide;

(e) N-(4-chlorobenzyl)-2-(2-morpholin-4-ylethyl)-8-(4-morpholinylmethyl)-6-oxo-6H-pyrrolo[3,2,1-ij]quinoline-5-carboxamide;

(f) N-(4-chlorobenzyl)-2-[2-(diethylamino)ethyl]-8-(4-morpholinylmethyl)-6-oxo-6H-pyrrolo[3,2,1-ij]quinoline-5-carboxamide;

(g) N-(4-chlorobenzyl)-2-[2-(4-methylpiperazin-1-yl)ethyl]-8-(morpholin-4-ylmethyl)-6-oxo-6H-pyrrolo[3,2,1-ij]quinoline-5-carboxamide;

(h) N-(4-chlorobenzyl)-2-[2-(2-ethylpiperidin-1-yl)ethyl]-8-(morpholin-4-ylmethyl)-6-oxo-6H-pyrrolo[3,2,1-ij]quinoline-5-carboxamide;

(i) N-(4-chlorobenzyl)-2-[3-(4-methylpiperazin-1-yl)propyl]-8-(morpholin-4-ylmethyl)-6-oxo-6H-pyrrolo[3,2,1-ij]quinoline-5-carboxamide;

(j) N-(4-chlorobenzyl)-8-(morpholin-4-ylmethyl)-6-oxo-2-(2-piperidin-1-ylethyl)-6H-pyrrolo[3,2,1-ij]quinoline-5-carboxamide;

(k) N-(4-chlorobenzyl)-8-(morpholin-4-ylmethyl)-2-(3-morpholin-4-ylpropyl)-6-oxo-6H-pyrrolo[3,2,1-ij]quinoline-5-carboxamide;

(l) N-(4-chlorobenzyl)-2-[(1,1-dioxido-4-thiomorpholinyl)methyl]-8-(4-morpholinylmethyl)-6-oxo-6H-pyrrolo[3,2,1-ij]quinoline-5-carboxamide;

(m) N-(4-chlorobenzyl)-2-[(1,1-dioxido-4-thiomorpholinyl)methyl]-1-iodo-8-(4-morpholinylmethyl)-6-oxo-6H-pyrrolo[3,2,1-ij]quinoline-5-carboxamide;

(n) N-(4-chlorobenzyl)-2-(3-hydroxypropyl)-8-(4-morpholinylmethyl)-6-oxo-6H-pyrrolo[3,2,1-ij]quinoline-5-carboxamide;

(o) 2-{[(aminocarbonyl)amino]methyl}-N-(4-chlorobenzyl)-8-(4-morpholinylmethyl)-6-oxo-6H-pyrrolo[3,2,1-ij]quinoline-5-carboxamide;

(p) N-(4-chlorobenzyl)-2-[(1R)-1-hydroxyethyl]-8-(4-morpholinylmethyl)-6-oxo-6H-pyrrolo[3,2,1-ij]quinoline-5-carboxamide;

(q) N-(4-chlorobenzyl)-2-(methoxymethyl)-8-(4-morpholinylmethyl)-6-oxo-6H-pyrrolo[3,2,1-ij]quinoline-5-carboxamide;

(r) N-(4-chlorobenzyl)-2-[(ethylsulfanyl)methyl]-8-(4-morpholinylmethyl)-6-oxo-6H-pyrrolo[3,2,1-ij]quinoline-5-carboxamide;

(s) N-(4-chlorobenzyl)-8-(4-morpholinylmethyl)-6-oxo-2-[(phenylsulfanyl)methyl]-6H-pyrrolo[3,2,1-ij]quinoline-5-carboxamide;

(t) N-(4-chlorobenzyl)-2-[(methylamino)methyl]-8-(4-morpholinylmethyl)-6-oxo-6H-pyrrolo[3,2,1-ij]quinoline-5-carboxamide;

(u) N-(4-chlorobenzyl)-2-[(dimethylamino)methyl]-8-(4-morpholinylmethyl)-6-oxo-6H-pyrrolo[3,2,1-ij]quinoline-5-carboxamide;

(v) N-(4-chlorobenzyl)-2-(2-hydroxyethyl)-6-oxo-6H-pyrrolo[3,2,1-ij]quinoline-5-carboxamide; or

(w) N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

A method of treating or preventing atherosclerosis or restenosis in a mammal, wherein the compound administered has the Formula II and is:

(a) N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide;

(b) N-(4-chlorobenzyl)-2-(hydroxymethyl)-8-(4-morpholinylmethyl)-6-oxo-6H-pyrrolo[3,2,1-ij]quinoline-5-carboxamide;

(c) N-(4-chlorobenzyl)-2-(2-hydroxyethyl)-8-(4-morpholinylmethyl)-6-oxo-6H-pyrrolo[3,2,1-ij]quinoline-5-carboxamide;

(d) N-(4-chlorobenzyl)-2-(2-morpholin-4-ylethyl)-8-(4-morpholinylmethyl)-6-oxo-6H-pyrrolo[3,2,1-ij]quinoline-5-carboxamide;

(e) N-(4-chlorobenzyl)-2-[(1,1-dioxido-4-thiomorpholinyl)methyl]-8-(4-morpholinylmethyl)-6-oxo-6H-pyrrolo[3,2,1-ij]quinoline-5-carboxamide;

(f) N-(4-chlorobenzyl)-2-[(1,1-dioxido-4-thiomorpholinyl)methyl]-1-iodo-8-(4-morpholinylmethyl)-6-oxo-6H-pyrrolo[3,2,1-ij]quinoline-5-carboxamide;

(g) N-(4-chlorobenzyl)-2-(3-hydroxypropyl)-8-(4-morpholinylmethyl)-6-oxo-6H-pyrrolo[3,2,1-ij]quinoline-5-carboxamide;

(h) 2-{[(aminocarbonyl)amino]methyl}-N-(4-chlorobenzyl)-8-(4-morpholinylmethyl)-6-oxo-6H-pyrrolo[3,2,1-ij]quinoline-5-carboxamide;

(i) N-(4-chlorobenzyl)-2-[(1R)-1-hydroxyethyl]-8-(4-morpholinylmethyl)-6-oxo-6H-pyrrolo[3,2,1-ij]quinoline-5-carboxamide;

(j) N-(4-chlorobenzyl)-2-(methoxymethyl)-8-(4-morpholinylmethyl)-6-oxo-6H-pyrrolo[3,2,1-ij]quinoline-5-carboxamide;

(k) N-(4-chlorobenzyl)-2-[(ethylsulfanyl)methyl]-8-(4-morpholinylmethyl)-6-oxo-6H-pyrrolo[3,2,1-ij]quinoline-5-carboxamide;

(l) N-(4-chlorobenzyl)-8-(4-morpholinylmethyl)-6-oxo-2-[(phenylsulfanyl)methyl]-6H-pyrrolo[3,2,1-ij]quinoline-5-carboxamide;

(m) N-(4-chlorobenzyl)-2-[(methylamino)methyl]-8-(4-morpholinylmethyl)-6-oxo-6H-pyrrolo[3,2,1-ij]quinoline-5-carboxamide;

(n) N-(4-chlorobenzyl)-2-[(dimethylamino)methyl]-8-(4-morpholinylmethyl)-6-oxo-6H-pyrrolo[3,2,1-ij]quinoline-5-carboxamide; or

(o) N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

A method of treating or preventing atherosclerosis or restenosis in a mammal, wherein the compound administered has the Formula II and is:

(gg) N-(4-chlorobenzyl)-2-(hydroxymethyl)-6-oxo-6H-pyrrolo[3,2,1-ij]quinoline-5-carboxamide;

(hh) N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide;

(ii) N-(4-chlorobenzyl)-2-(hydroxymethyl)-8-(4-morpholinylmethyl)-6-oxo-6H-pyrrolo[3,2,1-ij]quinoline-5-carboxamide;

(jj) N-(4-chlorobenzyl)-2-(2-hydroxyethyl)-8-(4-morpholinylmethyl)-6-oxo-6H-pyrrolo[3,2,1-ij]quinoline-5-carboxamide;

(kk) N-(4-chlorobenzyl)-2-(2-morpholin-4-ylethyl)-8-(4-morpholinylmethyl)-6-oxo-6H-pyrrolo[3,2,1-ij]quinoline-5-carboxamide;

(ll) N-(4-chlorobenzyl)-2-[2-(diethylamino)ethyl]-8-(4-morpholinylmethyl)-6-oxo-6H-pyrrolo[3,2,1-ij]quinoline-5-carboxamide;

(mm) N-(4-chlorobenzyl)-2-[2-(4-methylpiperazin-1-yl)ethyl]-8-(morpholin-4-ylmethyl)-6-oxo-6H-pyrrolo[3,2,1-ij]quinoline-5-carboxamide;

(nn) N-(4-chlorobenzyl)-2-[2-(2-ethylpiperidin-1-yl)ethyl]-8-(morpholin-4-ylmethyl)-6-oxo-6H-pyrrolo[3,2,1-ij]quinoline-5-carboxamide;

(oo) N-(4-chlorobenzyl)-2-[3-(4-methylpiperazin-1-yl)propyl]-8-(morpholin-4-ylmethyl)-6-oxo-6H-pyrrolo[3,2,1-ij]quinoline-5-carboxamide;

(pp) N-(4-chlorobenzyl)-8-(morpholin-4-ylmethyl)-6-oxo-2-(2-piperidin-1-ylethyl)-6H-pyrrolo[3,2,1-ij]quinoline-5-carboxamide;

(qq) N-(4-chlorobenzyl)-8-(morpholin-4-ylmethyl)-2-(3-morpholin-4-ylpropyl)-6-oxo-6H-pyrrolo[3,2,1-ij]quinoline-5-carboxamide;

(rr) N-(4-chlorobenzyl)-2-[(1,1-dioxido-4-thiomorpholinyl)methyl]-8-(4-morpholinylmethyl)-6-oxo-6H-pyrrolo[3,2,1-ij]quinoline-5-carboxamide;

(ss) N-(4-chlorobenzyl)-2-[(1,1-dioxido-4-thiomorpholinyl)methyl]-1-iodo-8-(4-morpholinylmethyl)-6-oxo-6H-pyrrolo[3,2,1-ij]quinoline-5-carboxamide;

(tt) N-(4-chlorobenzyl)-2-(3-hydroxypropyl)-8-(4-morpholinylmethyl)-6-oxo-6H-pyrrolo[3,2,1-ij]quinoline-5-carboxamide;

(uu) 2-{[(aminocarbonyl)amino]methyl}-N-(4-chlorobenzyl)-8-(4-morpholinylmethyl)-6-oxo-6H-pyrrolo[3,2,1-ij]quinoline-5-carboxamide;

(vv) N-(4-chlorobenzyl)-2-[(1R)-1-hydroxyethyl]-8-(4-morpholinylmethyl)-6-oxo-6H-pyrrolo[3,2,1-ij]quinoline-5-carboxamide;

(ww) N-(4-chlorobenzyl)-2-(methoxymethyl)-8-(4-morpholinylmethyl)-6-oxo-6H-pyrrolo[3,2,1-ij]quinoline-5-carboxamide;

(xx) N-(4-chlorobenzyl)-2-[(ethylsulfanyl)methyl]-8-(4-morpholinylmethyl)-6-oxo-6H-pyrrolo[3,2,1-ij]quinoline-5-carboxamide;

(yy) N-(4-chlorobenzyl)-8-(4-morpholinylmethyl)-6-oxo-2-[(phenylsulfanyl)methyl]-6H-pyrrolo[3,2,1-ij]quinoline-5-carboxamide;

(zz) N-(4-chlorobenzyl)-2-[(methylamino)methyl]-8-(4-morpholinylmethyl)-6-oxo-6H-pyrrolo[3,2,1-ij]quinoline-5-carboxamide;

(aaa) N-(4-chlorobenzyl)-2-[(dimethylamino)methyl]-8-(4-morpholinylmethyl)-6-oxo-6H-pyrrolo[3,2,1-ij]quinoline-5-carboxamide;

(bbb) N-(4-chlorobenzyl)-2-(2-hydroxyethyl)-6-oxo-6H-pyrrolo[3,2,1-ij]quinoline-5-carboxamide;

(ccc) N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide;

(ddd) N-(4-chlorobenzyl)-2-[2-hydroxy-1-(hydroxymethyl)ethyl]-8-(4-morpholinylmethyl)-6-oxo-6H-pyrrolo[3,2,1-ij]quinoline-5-carboxamide;

(eee) N-(4-chlorobenzyl)-2-[1,2-dihydroxy-1-(hydroxymethyl)ethyl]-8-(4-morpholinylmethyl)-6-oxo-6H-pyrrolo[3,2,1-ij]quinoline-5-carboxamide;

(fff) N-(4-chlorobenzyl)-2-[(ethylsulfonyl)methyl]-8-(4-morpholinylmethyl)-6-oxo-6H-pyrrolo[3,2,1-ij]quinoline-5-carboxamide;

(ggg) N-(4-chlorobenzyl)-2-[(ethylsulfinyl)methyl]-8-(4-morpholinylmethyl)-6-oxo-6H-pyrrolo[3,2,1-ij]quinoline-5-carboxamide;

(hhh) 2-{[bis(2-hydroxyethyl)amino]methyl}-N-(4-chlorobenzyl)-8-(4-morpholinylmethyl)-6-oxo-6H-pyrrolo[3,2,1-ij]quinoline-5-carboxamide;

(iii) N-(4-chlorobenzyl)-2-[(2-hydroxyethoxy)methyl]-8-(4-morpholinylmethyl)-6-oxo-6H-pyrrolo[3,2,1-ij]quinoline-5-carboxamide;

(jjj) N-(4-chlorobenzyl)-2-(1,2-dihydroxyethyl)-8-(4-morpholinylmethyl)-6-oxo-6H-pyrrolo[3,2,1-ij]quinoline-5-carboxamide;

(kkk) N-(4-chlorobenzyl)-8-(4-morpholinylmethyl)-6-oxo-2-(1,2,3-trihydroxypropyl)6H-pyrrolo[3,2,1-ij]quinoline-5-carboxamide;

(lll) N-(4-chlorobenzyl)-2-[3-hydroxy-2-(hydroxymethyl)propyl]-8-(4-morpholinylmethyl)-6-oxo-6H-pyrrolo[3,2,1-ij]quinoline-5-carboxamide;

(mmm) N-(4-chlorobenzyl)-1-(hydroxymethyl)-8-(morpholin-4-ylmethyl)-6-oxo-6H-pyrrolo[3,2,1-ij]quinoline-5-carboxamide;

(nnn) N-(4-chlorobenzyl)-1-(2-hydroxyethyl)-8-(morpholin-4-ylmethyl)-6-oxo-6H-pyrrolo[3,2,1-ij]quinoline-5-carboxamide;

(ooo) N-(4-chlorobenzyl)-1-(3-hydroxypropyl)-8-(morpholin-4-ylmethyl)-6-oxo-6H-pyrrolo[3,2,1-ij]quinoline-5-carboxamide;

(ppp) N-(4-chlorobenzyl)-1-(2-morpholin-4-ylethyl)-8-(morpholin-4-ylmethyl)-6-oxo-6H-pyrrolo[3,2,1-ij]quinoline-5-carboxamide;

(qqq) N-(4-chlorobenzyl)-8-(morpholin-4-ylmethyl)-6-oxo-1-(2-thiomorpholin-4-ylethyl)-6H-pyrrolo[3,2,1-ij]quinoline-5-carboxamide;

(rrr) N-(4-chlorobenzyl)-1-[2-(1,1-dioxidothiomorpholin-4-yl)ethyl]-8-(morpholin-4-ylmethyl)-6-oxo-6H-pyrrolo[3,2,1-ij]quinoline-5-carboxamide;

(sss) N-(4-chlorobenzyl)-1-[2-(4-methylpiperazin-1-yl)ethyl]-8-(morpholin-4-ylmethyl)-6-oxo-6H-pyrrolo[3,2,1-ij]quinoline-5-carboxamide;

(ttt) N-(4-chlorobenzyl)-8-(morpholin-4-ylmethyl)-6-oxo-1-(2-piperazin-1-ylethyl)-6H-pyrrolo[3,2,1-ij]quinoline-5-carboxamide;

(uuu) 1-[(acetylamino)methyl]-N-(4-chlorobenzyl)-8-(morpholin-4-ylmethyl)-6-oxo-6H-pyrrolo[3,2,1-ij]quinoline-5-carboxamide;

(vvv) N-(4-chlorobenzyl)-1-[(1S)-1-hydroxyethyl]-8-(morpholin-4-ylmethyl)-6-oxo-6H-pyrrolo[3,2,1-ij]quinoline-5-carboxamide;

(www) N-(4-chlorobenzyl)-1-(1H-imidazol-1-ylmethyl)-8-(morpholin-4-ylmethyl)-6-oxo-6H-pyrrolo[3,2,1-ij]quinoline-5-carboxamide;

(xxx) 1-(1H-1,2,3-benzotriazol-1-ylmethyl)-N-(4-chlorobenzyl)-8-(morpholin-4-ylmethyl)-6-oxo-6H-pyrrolo[3,2,1-ij]quinoline-5-carboxamide;

(yyy) N-(4-chlorobenzyl)-8-(morpholin-4-ylmethyl)-6-oxo-1-(pyridin-3-ylmethyl)-6H-pyrrolo[3,2,1-ij]quinoline-5-carboxamide;

(zzz) N-(4-chlorobenzyl)-8-(morpholin-4-ylmethyl)-6-oxo-1-{[({[3-(trifluoromethyl)phenyl]amino}carbonyl)amino]methyl}-6H-pyrrolo[3,2,1-ij]quinoline-5-carboxamide;

(aaaa) N-(4-chlorobenzyl)-8-(4-morpholinylmethyl)-6-oxo-2-[2-(3-oxo-1-azetidinyl)ethyl]-6H-pyrrolo[3,2,1-ij]quinoline-5-carboxamide;

(bbbb) N-(4-chlorobenzyl)-2-[2-(3-hydroxy-1-azetidinyl)ethyl]-8-(4-morpholinylmethyl)-6-oxo-6H-pyrrolo[3,2,1-ij]quinoline-5-carboxamide;

(cccc) N-(4-chlorobenzyl)-2-(2,3-dihydroxypropyl)-8-(4-morpholinylmethyl)-6-oxo-6H-pyrrolo[3,2,1-ij]quinoline-5-carboxamide;

(dddd) N-(4-chlorobenzyl)-2-[(1S)-1-hydroxyethyl]-8-(4-morpholinylmethyl)-6-oxo-6H-pyrrolo[3,2,1-ij]quinoline-5-carboxamide;

(eeee) N-(4-chlorobenzyl)-2-[2-(1H-imidazol-1-yl)ethyl]-8-(morpholin-4-ylmethyl)-6-oxo-6H-pyrrolo[3,2,1-ij]quinoline-5-carboxamide;

(ffff) N-(4-chlorobenzyl)-2-[2-(1H-imidazol-2-yl)ethyl]-8-(morpholin-4-ylmethyl)-6-oxo-6H-pyrrolo[3,2,1-ij]quinoline-5-carboxamide;

(gggg) N-(4-chlorobenzyl)-8-(morpholin-4-ylmethyl)-6-oxo-2-[2-(4H-1,2,4-triazol-3-yl)ethyl]-6H-pyrrolo[3,2,1-ij]quinoline-5-carboxamide;

(hhhh) N-(4-chlorobenzyl)-8-(morpholin-4-ylmethyl)-6-oxo-2-[2-(1H-tetraazol-5-yl)ethyl]-6H-pyrrolo[3,2,1-ij]quinoline-5-carboxamide;

(iiii) N-(4-chlorobenzyl)-8-(morpholin-4-ylmethyl)-6-oxo-2-(2-piperazin-1-ylethyl)-6H-pyrrolo[3,2,1-ij]quinoline-5-carboxamide; or

(jjjj) tert-butyl 4-{2-[5-{[(4-chlorobenzyl)amino]carbonyl}-8-(morpholin-4-ylmethyl)-6-oxo-6H-pyrrolo[3,2,1-ij]quinolin-2-yl]ethyl}piperazine-1-carboxylate, or a pharmaceutically acceptable salt thereof.

A method of treating or preventing atherosclerosis or restenosis in a mammal, wherein the compound administered has the Formula II and is N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

A method of treating or preventing atherosclerosis or restenosis in a mammal, wherein the compound administered has the Formula II and is N-(4-chlorobenzyl)-2-(hydroxymethyl)-8-(4-morpholinylmethyl)-6-oxo-6H-pyrrolo[3,2,1-ij]quinoline-5-carboxamide, or a pharmaceutically acceptable salt thereof.

A method of treating or preventing atherosclerosis or restenosis in a mammal, wherein the compound administered has the Formula II and is N-(4-chlorobenzyl)-2-(2-hydroxyethyl)-8-(4-morpholinylmethyl)-6-oxo-6H-pyrrolo[3,2,1-ij]quinoline-5-carboxamide, or a pharmaceutically acceptable salt thereof.

A method of treating or preventing atherosclerosis or restenosis in a mammal, wherein the compound administered has the Formula II and is N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

Also provided is the use of compounds of Formulae I, I′ and II to prepare a medicament for treating or preventing atherosclerosis or restenosis in a mammal.

Dosages and Dosage Forms

By the term “effective amount” of a compound as provided herein is meant a nontoxic but sufficient amount of one or more anti-atherosclerosis or anti-restenosis agents to provide the desired effect. The desired effect may be to prevent, give relief from, or ameliorate atherosclerosis or restenosis.

As pointed out below, the exact amount of the anti-atherosclerosis or anti-restenosis agent required to treat atherosclerosis or restenosis will vary from subject to subject, depending on the species, age, and general condition of the subject, the severity of the disease that is being treated, the particular compound(s) used, the mode of administration, such as the route and frequency of administration, and the particular compound(s) employed, and the like. Thus, it is not possible to specify an exact “effective amount.” However, an appropriate effective amount may be determined by one of ordinary skill in the art using only routine experimentation.

Pharmaceutical compositions including one or more anti-atherosclerosis or anti-restenosis agents of Formula I-V or XI can be administered orally or parenterally at dose levels, calculated as the free base, of each of the anti-atherosclerosis or anti-restenosis agent at 0.1 to 300 mg/kg of mammal body weight, preferably 1.0 to 30 mg/kg of mammal body weight, and can be used in a human in a unit dosage form, administered one to four times daily in the amount of 1 to 1000 mg per unit dose. The desired dosage may conveniently be presented in a single dose or as divided into multiple doses administered at appropriate intervals, for example, as two, three, four or more sub-doses per day. The sub-dose itself may be further divided, e.g., into a number of discrete loosely spaced aministrations.

Initial treatment of a patient suffering from atherosclerosis or restenosis can begin with a dosage regimen as indicated above. Treatment is generally continued as necessary over a period of several weeks to several months or years until the condition or disorder has been controlled or eliminated. Patents undergoing treatment with a composition of the invention can be routinely monitored by any of the methods well known in the art to determine the effectiveness of therapy. Continuous analysis of data from such monitoring permits modification of the treatment regin during therapy so that optimally effective amounts of drug are administered at any point in time, and so that the duration of treatment can be determined. In this way, the treatment regimen and dosing schedule can be rationally modified over the course of therapy so that the lowest amount of the compounds of this invention exhibiting satisfactory effectiveness is administered, and so that administration is continued only for so long as is necessary to successfully treat the condition or disorder.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

In a combination therapy, the anti-atherosclerosis or anti-restenosis agent compound(s) and other inhibitor compound(s) can be administered simultaneously or at separate intervals. When administered simultaneously the anti-atherosclerosis or anti-restenosis agent compound(s) and the other inhibitor compound(s) can be incorporated into a single pharmaceutical composition or into separate compositions, e.g., anti-atherosclerosis or anti-restenosis agent compound(s) in one composition and the other inhibitor compound(s) in another composition. For instance the combination therapy, the anti-atherosclerosis or anti-restenosis agent compound(s) may be administered concurrently or concomitantly with the other inhibitor compound(s). The term “concurrently” means the subject being treated takes one drug within about 5 minutes of taking the other drug. The term “concomitantly” means the subject being treated takes one drug within the same treatment period of taking the other drug. The same treatment period is preferably within twelve hours and up to forty-eight hours.

When separately administered, therapeutically effective amounts of anti-atherosclerosis or anti-restenosis agent compound(s) and the other inhibitor compound(s) are administered on a different schedule. One may be administered before the other as long as the time between the two administrations falls within a therapeutically effective interval. A therapeutically effective interval is a period of time beginning when one of either (a) the anti-atherosclerosis or anti-restenosis agent compound(s), or (b) the other inhibitor compound(s) is administered to a mammal and ending at the limit of the beneficial effect in the treatment of atherosclerosis or restenosis of the combination of (a) and (b). The methods of administration of the anti-atherosclerosis or anti-restenosis agent compound(s) and the other inhibitor compound(s) may vary. Thus, one agent may be administered orally, while the other is administered by injection.

A specific active agent may have more than one recommended dosage range, particularly for different routes of administration. Generally, an effective amount of dosage of anti-atherosclerosis or anti-restenosis agent compound(s), either administered individually or in combination with other inhibitor compound(s), will be in the range of about 0.1 to about 300 mg/kg of body weight/day, preferably about 1 to about 30 mg/kg of body weight/day. It is to be understood that the dosages of active component(s) may vary depending upon the requirements of each subject being treated and the severity of the atherosclerosis or restenosis.

In addition to the anti-atherosclerosis or anti-restenosis agents, the composition for therapeutic use may also comprise one or more non-toxic, pharmaceutically acceptable carrier materials or excipients. The term carrier material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion or active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Generally, the concentration of each of the anti-atherosclerosis or anti-restenosis agents in a liquid composition, such as a lotion, will be from about 0.1 wt. % to about 20 wt. %, preferably from about 0.5 wt. % to about 10 wt. %. The solution may contain other ingredients, such as emulsifiers, antioxidants or buffers. The concentration in a semi-solid or solid composition, such as a gel or a powder, will be about 0.1 wt. % to about 5 wt. %, preferably about 0.5 wt. % to about 2.5 wt. %. When the topically deliverable, pharmaceutical composition of the present invention is utilized to effect targeted treatment of a specific internal site, each of the anti-atherosclerosis or anti-restenosis agent is preferably contained in the composition in an amount of from 0.05-10 wt. %., more preferably 0.5-5 wt. %.

Routes of Administration

In therapeutic use for treating, or combating, viral infections in a mammal (i.e., human and animals) the pharmaceutical composition including the anti-atherosclerosis or anti-restenosis agent(s) can be administered orally, parenterally, topically, rectally, or intranasally.

Parenteral administrations include injections to generate a systemic effect or injections directly to the afflicted ara. Examples to parenteral administrations are subcutaneous, intravenous, intramuscular, intradermal, intrathecal, intraocular, intraventricular, and general infusion techniques.

Topical administrations includes transdermal delivery to generate a system effect.

The rectal administration includes the form of suppositories.

The intranasally administration includes nasal aerosol or inhalation applications.

Pharmaceutical compositions including the anti-atherosclerosis or anti-restenosis agent(s) may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the anti-atherosclerosis or anti-restenosis agent(s) can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a bonder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of pharmaceutical compositions with the anti-atherosclerosis or anti-restenosis agent(s) dissolved in water and water-propylene glycol and water-polyethylene glycol systems, optionally containing suitable conventional coloring agents, flavoring agents, stabilizers and thickening agents.

The anti-atherosclerosis or anti-restenosis agent(s) may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oil or aqueous vehicles, and may contain formulating materials such as suspending, stabilizing and/or dispersing agents.

For injection, the anti-atherosclerosis or anti-restenosis agent(s) may be formulated in aqueous solution, preferably in physiologically compatible buffers or physiological saline buffer. Suitable buffering agents include tri-sodium orthophosphate, sodium bicarbonate, sodium citrate, N-methyl-glucamine, L(+)-lysine and L(+)-arginine.

The compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Other parenteral administrations also include aqueous solutions of a water soluble form, such as, without limitation, a salt, of the anti-atherosclerosis or anti-restenosis agent(s). Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the anti-atherosclerosis or anti-restenosis agent(s) may be in a powder form for constitution with a suitable vehicle, e.g., sterile, pyrogen-free water, before use.

For suppository administration, the pharmaceutical compositions may also be formulated by mixing the anti-atherosclerosis or anti-restenosis agent(s) with a suitable non-irritating excipient which is solid at room temperature but liquid at rectal temperature and therefore will melt in the rectum to release the drug. Such materials include cocoa butter, beeswax and other glycerides.

For administration by inhalation, the anti-atherosclerosis or anti-restenosis agent(s) can be conveniently delivered through an aerosol spray in the form of solution, dry powder, or cream. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a powder base such as lactose or starch.

In addition to the formulations described previously, the anti-atherosclerosis or anti-restenosis agent(s) may also be formulated as depot preparations. Such long acting formulations may be in the form of implants. The anti-atherosclerosis or anti-restenosis agent(s) may be formulated for this route of administration with suitable polymers, hydrophobic materials, or as a sparing soluble derivative such as, without limitation, a sparingly soluble salt.

Additionally, the anti-atherosclerosis or anti-restenosis agent(s) may be delivered using a sustained-release system. Various sustained-release materials have been estabilshed and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

In certain embodiments, the anti-atherosclerosis or anti-restenosis agent(s) are applied topically. For topical applications, the pharmaceutical composition may be formulated in a suitable ointment containing the anti-atherosclerosis or anti-restenosis agent(s) suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

Several different animal models are available to evaluate reduction of atherosclerosis or restinosis by antiviral drug treatment. In these models histological changes in the atherosclerotic lesions of aortic arteries are measured in animals infected with a herpesvirus and treated or untreated with an antiviral drug capable of inhibiting replication of the herpesvirus. The models include murine CMV infection of apoE deficient mice and rat CMV infection of rats. These models would mimic the effects of human CMV infection. MHV-68 is a murine gammaherpesvirus related to EBV. Antiviral treatment has been shown to reduce atherosclerosis caused by HMV-68 infection in apoE deficient mice. Drugs containing compounds of Formula I and II inhibit replication of these animal viruses so the models could be used to show an effect of drugs containing compounds of Formula I and II on development of atherosclerosis. Lemstrom, et al, ¢Cytomegalovirus Infection-Enhanced Allograft Atherosclerosis is prevented by DHPG Prophylaxis in the Rat”, Circulation Vol. 90, No. 4, October 1994, pp 1969-1978; Burnell et al, “Atherosclerosis in a poE Knockout Mice Infected with Multiple Pathogens”. Both of these references are herein incorporated by reference.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited by the claims which follow.

All published documents are incorporated by reference herein.

What is claimed is:

1. A method of treating atherosclerosis or restenosis in a mammal, comprising administering to said mammal an effective amount of a compound selected from the group consisting of structures of Formulae I, I′ and II, wherein Formula I is 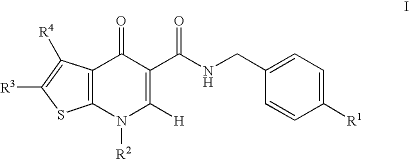
or a pharmaceutically acceptable salt, racemate, solvate, tautomer, optical isomer or prodrug derivative thereof, wherein,
R¹ is
(a) Cl,
(b) Br,
(c) CN,
(d) NO₂, or
(e) F;
R² is
(a) H,
(b) R⁵,
(c) NR⁷R⁸,
(d) SO₂R⁹, or
(e) OR⁹;
R³ is
(a) H,
(b) halo,
(c) aryl,
(d) S(O)ₘR⁶,
(e) (C═O)R⁶,
(f) (C═O)OR⁹,
(g) cyano,
(h) het, wherein said het is bound via a carbon atom,
(i) OR¹⁰,
(j) Ohet,
(k) NR⁷R⁸
(l) SR¹⁰,
(m) Shet,
(n) NHCOR¹²,
(o) NHSO₂R¹², or
(p) C₁₋₇alkyl which may be partially unsaturated and optionally substituted by one or more substituents of the group R¹¹, OR¹³, SR¹⁰, SR¹³, NR⁷R⁸, halo, (C═O)C₁₋₇alkyl, or SOₘR⁹;
R⁴ is
(a) H,
(b) halo,
(c) C₁₋₄alkyl, or
(d) R⁴ together with R³ form a carbocyclic or het, either of which may be optionally substituted by NR⁷R⁸, by C₁₋₇alkyl which may be optionally substituted by OR¹⁴, or by het, wherein said het is bound via a carbon atom;
R⁵ is
(a) (CH₂CH₂O)ᵢR¹⁰,
(b) het, wherein said het is bound via a carbon atom,
(c) aryl,
(d) C₁₋₇alkyl which may be partially unsaturated and is optionally substituted by one or more substituents selected from a group consisting of NR⁷R⁸, R¹¹, SOₘR⁹, and OC₂₋₄alkyl which may be further substituted by het, OR¹⁰, or NR⁷R⁸, or
(e) C₃₋₈cycloalkyl which may be partially unsaturated and optionally substituted by one or more substituents selected from a group consisting of R¹¹, NR⁷R⁸, SOₘR⁹, and C₁₋₇alkyl optionally substituted by R¹¹, NR⁷P⁸, or SOₘR⁹;
R⁶ is
(a) C₁₋₇alkyl,
(b) NR⁷R⁸,
(c) aryl, or
(d) het, wherein said het is bound via a carbon atom;
R⁷ and R⁸ are independently
(a) H,
(b) aryl,
(c) C₁₋₇alkyl which may be partially unsaturated and is optionally substituted by one or more substituents selected from a group consisting of NR¹⁰R¹⁰, R¹¹, SOₘR⁹, CONR¹⁰R¹⁰, or halo, or,
(d) R⁷ and R⁸ together with the nitrogen to which they are attached form a het;
R⁹ is
(a) aryl,
(b) het,
(c) C₃₋₈cycloalkyl, or
(d) C₁₋₇alkyl which may be partially unsaturated and is optionally substituted by one or more substituents selected from a group consisting of NR¹⁰R¹⁰, R¹¹, SH, CONR¹⁰R¹⁰, or halo;
R¹⁰ is
(a) H, or
(b) C₁₋₇alkyl optionally substituted by OH;
R¹¹ is
(a) OR¹⁰,
(b) Ohet,
(c) Oaryl,
(d) CO₂R¹⁰,
(e) het,
(f) aryl, or
(g) CN;
R¹² is
(a) H,
(b) het,
(c) aryl,
(d) C₃₋₈cycloalkyl, or
(e) C₁₋₇alkyl optionally substituted by NR⁷R⁸ or R¹¹;
R¹³ is
(a) (P═O)(OR¹⁴)₂,
(b) CO(CH₂)ₙCON(CH₃)—(CH₂)ₙSO₃⁻M⁺,
(c) an amino acid,
(d) C(═O)aryl, or
(e) C(═O)C₁₋₇alkyl optionally substituted by NR⁷R⁸, aryl, het, CO₂H, or O(CH₂)ₙCO₂R¹⁴);
R¹⁴ is
(a) H, or
(b) C₁₋₇alkyl;
each i is independently 2, 3, or 4;
each n is independently 1, 2, 3, 4 or 5;
each m is independently 0, 1, or 2; and
M is sodium, potassium, or lithium;
wherein any aryl is optionally substituted with one or more substituents selected from the group consisting of halo, OH, cyano, CO₂R¹⁴, CF₃, C₁₋₆alkoxy, and C₁₋₆ alkyl which maybe further substituted by one to three SR¹⁴, NR¹⁴R¹⁴, OR¹⁴, het, and CO₂R¹⁴; and
wherein any het is optionally substituted with one or more substituents selected from the group consisting of halo, OH, cyano, phenyl, CO₂R¹⁴, CF₃, C₁₋₆alkoxy, oxo, oxime, and C₁₋₆ alkyl which maybe further substituted by one to three SR¹⁴, NR¹⁴R¹⁴, OR¹⁴, and CO₂R¹⁴.
aryl, in Formula I, denotes a phenyl radical or an ortho-fused bicyclic carbocyclic radical having about nine to ten ring atoms in which at least one ring is aromatic. Het is a four-(4), five-(5), six-(6), or seven-(7) membered saturated or unsaturated heterocyclic ring having 1, 2, 3, or 4 heteroatoms selected from the group consisting of oxy, thio, sulfinyl, sulfonyl, and nitrogen, which is optionally fused to a benzene ring, or any bicyclic heterocycle group;
het, in Formula I, includes “heteroaryl,” which encompasses a radical attached via a ring carbon of a monocyclic aromatic ring containing five or six ring atoms consisting of carbon and 1, 2, 3, or 4 heteroatoms each selected from the group consisting of non-peroxide oxy, thio, and N(X) wherein X is absent or is H, O, C₁₋₄alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto; and wherein Formula I′ is 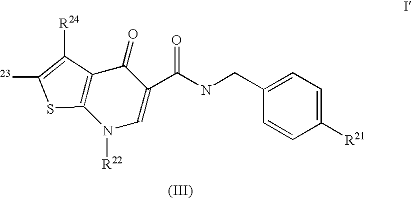
or a pharmaceutically acceptable salt, racemate, solvate, tautomer, optical isomer or prodrug derivative thereof wherein,
R²¹ is Cl, Br, CN, or NO₂;
R²² is H, —(CH₂CH₂O)ₙH, —(CH₂CH₂O)ₙCH₃, SO₂R³⁵ or COR³⁵, C₁₋₇alkyl which may be partially unsaturated and optionally substituted by R³⁶, C₂₋₇alkyl which may be partially unsaturated and optionally substituted by R³³, or C₃₋₈cycloalkyl which may be partially unsaturated and optionally substituted by R³⁶, R³³ or R³⁴;
each R²³ and R²⁴ is independently H, halo, aryl, S(O)ₘR³⁰, COR³⁰, cyano, het, CF₃, OR²⁹, OR³¹, SR²⁹, SR³¹, NR²⁵R²⁶, CH(OR²⁹)R²⁷, CO₂R²⁹, CH(CO₂R²⁹)₂, NHCOR²⁷, or NHS(O)₂R²⁷ or C₁₋₇alkyl which may be partially unsaturated and optionally substituted by R²⁸;
each R²⁵ and R²⁶ is independently H or C₁₋₇alkyl;
R²⁷ is C₁₋₇alkyl optionally substituted by R³⁶ or C₂₋₇alkyl optionally substituted by R³³;
R²⁸ is cyano, halo, CF₃, aryl, het, C(═O)C₁₋₇ alkyl, CO₂C₁₋₇alkyl, OR²⁹, OR³¹, OR³², SR²⁹, SR³¹, SR³², NR²⁵R²⁶, CH(OR²⁹)R²⁷, CO₂R²⁹ or CH(CO₂R²⁹)₂;
R²⁹ is H or C₁₋₇alkyl;
R³⁰ is C₁₋₇alkyl, NR²⁵R²⁶, aryl or het;
R³¹ is C₂₋₇alkyl substituted by OH;
R³² is (P═O)(OR²⁹)₂, CO(CH₂)ₙCON(CH₃)—(CH₂)ₙSO₃⁻M⁺, an amino acid, C(═O)aryl, or C(═O)C₁₋₇alkyl optionally substituted by NR²⁵R²⁶, aryl, het, carboxy, or O(CH₂)ₙCO₂R²⁹;
R³³ is hydroxy or NR²⁵R²⁶;
R³⁴ is C₁₋₇alkyl optionally substituted R³³;
R³⁵ is C₁₋₇alkyl, aryl or het;
R³⁶ is CO₂H or CO₂C₁₋₇alkyl
each n is independently 1, 2, 3, 4, or 5;
each m is independently 0, 1, or 2;
M is a pharmaceutically acceptable cation (e.g. sodium, potassium, or lithium);
wherein any aryl, or het in Formula I′ is optionally substituted with one or more substituents (e.g. 1, 2, 3, 4, or 5) independently selected from the group consisting of halo, cyano, trifluoromethyl, trifluoromethoxy, hydroxy, carboxy, OR²⁷, phenyl, phenoxy, (C₁₋₇alkoxy)carbonyl, SR³¹, and C₁₋₇ alkyl optionally substituted with one or more substituents independently selected from the group consisting of cyano, aryl, mercapto, het, R³⁶, OR²⁷, SR²⁷, and SR³¹; wherein phenyl or phenoxy is optionally substituted with one or more substituents independently selected from cyano, halo, trifluoromethyl, trifluoromethoxy, carboxy, het, OR³¹, and R²⁷,
wherein Formula II is 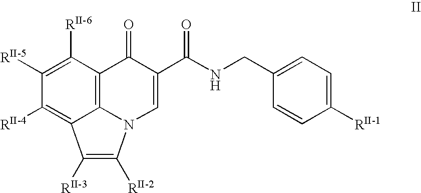
or a pharmaceutically acceptable salt, racemate, solvate, tautomer, optical isomer or prodrug derivative thereof wherein:
R^{II-1} is
(a) F,
(b) Cl,
(c) Br,
(d) CN or
(e) NO₂;
R^{II-2} and R^{II-3} are independently
(a) H,
(b) halo,
(c) OR^{II-11},
(d) C(═O)R^{II-7},
(e) C(═O)OR^{II-7},
(f) C₃₋₈cycloalkyl, or
(g) C₁₋₇alkyl which may be partially unsaturated and optionally substituted by one or more halo, C₃₋₈-cycloalkyl, R^{II-12}, OR^{II-14}, SR^{II-11}, SR^{II-14}, NR^{II-8}R^{II-9}, NR^{II-11}C(O)R^{II-7}, (C═O)C₁₋₇ alkyl, or SOₘR^{II-10};
R^{II-4}, and R^{II-5} are independently
(a) H,
(b) halo,
(c) aryl,
(d) S(O)ₘR^{II-7},
(e) (C═O)R^{II-7},
(f) (C═O)OR^{II-10},
(g) CN,
(h) het, wherein said het is bound via a carbon atom,
(i) OR^{II-11},
(j) Ohet,
(k) NR^{II-8}R^{II-9}
(l) SR^{II-11},
(m) Shet,
(n) NHCOR^{II-13},
(o) NHSO₂R^{II-13},
(p) C₃₋₈cycloalkyl, or
(q) C₁₋₇alkyl which may be partially unsaturated and optionally substituted by one or more R^{II-12}, OR^{II-14}, SR^{II-11}, SR^{II-14}, NR^{II-8}R^{II-9}, halo, C₃₋₈cycloalkyl, (C═O)C₁₋₇alkyl, or SOₘR^{II-10};
R^{II-6} is
(a) H,
(b) halo,
(c) C₃₋₈cycloalkyl, or
(d) C₁₋₄alkyl optionally substituted by 1-3 halo;
R^{II-7} is
(a) C₁₋₇alkyl,
(b) C₃₋₈cycloalkyl,
(c) NR^{II-8}R^{II-9},
(d) aryl, or
(e) het, wherein said het is bonded via a carbon atom;
R^{II-8} and R^{II-9} are independently
(a) H,
(b) aryl,
(c) C₃₋₈cycloalkyl,
(d) C₁₋₇alkyl which may be partially unsaturated and is optionally substituted by one or more NR^{II-11}R^{II-1}, R^{II-12}, SOₘR^{II-10}, CONR^{II-11}R^{II-11}, OH, aryl, het, C₃₋₈cycloalkyl, or halo, or
(e) R^{II-8} and R^{II-9} together with the nitrogen to which they are attached for a het;
R^{II-10} is
(a) aryl,
(b) het,
(c) C₃₋₈cycloalkyl, or
(d) C₁₋₇alkyl which may be partially unsaturated and is optionally substituted by one or more NR^{II-11}R^{II-11}, R^{II-12}, SH, CONR^{II-11}R^{II-11}, C₃₋₈cycloalkyl, or halo;
R^{II-11} is
(a) H,
(b) aryl,
(c) C₃₋₈cycloalkyl, or
(d) C₁₋₇alkyl optionally substituted by OH;
R^{II-12} is
(a) OR^{II-11},
(b) Ohet,
(c) Oaryl,
(d) CO₂R^{II-11},
(e) het,
(f) aryl, or
(g) CN;
R^{II-13} is
(a) H,
(b) het,
(c) aryl,
(d) C₃₋₈cycloalkyl, or
(e) C₁₋₇alkyl optionally substituted by NR^{II-11}R^{II-11} or R^{II-12};
R^{II-14} is
(a) (P═O)(OR^{II-15})₂,
(b) CO (CH₂)ₙCON(CH₃)—(CH₂)ₙSO₃⁻M⁺,
(c) an amino acid,
(d) C(═O)aryl, or
(e) C(═O)C₁₋₇alkyl optionally substituted by NR^{II-11}R^{II-11}, aryl, het, CO₂H, or O(CH₂)ₙCO₂R^{II-15};
R^{II-15} is
(a) H, or
(b) C₁₋₇alkyl;
aryl, in Formula II, is a phenyl radical or an ortho-fused bicyclic carbocyclic radical wherein at least one ring is aromatic; at each occurrence, aryl may be additionally substituted with one or more halo, CN, CO₂R^{II-11}, SR^{II-11}, OR^{II-11}, NR^{II-11}R^{II-11}, C₁₋₄alkyl, CF₃, or C₃₋₈cycloalkyl;
het, in Formula II, is a four-(4), five-(5), six-(6), or seven-(7) membered saturated or unsaturated heterocyclic ring having 1, 2, or 3 heteroatoms selected from the group consisting of O, SOₘ, and NX; wherein X is H, C₁₋₄alkyl or absence, wherein het is optionally fused to a benzene ring, or any bicyclic heterocycle group; at each occurrence, het may be additionally substituted with one or more halo, CN, CO₂R^{II-11}, SR^{II-11}, OR^{II-11}, NR^{II-11}R^{II-11}, C(═O) R^{II-13}, C₁₋₄alkyl, CF₃, C₃₋₈cycloalkyl, oxo or oxime;
at each occurrence, m is independently 0, 1, or 2;
at each occurrence, n is independently 1, 2, 3, 4, 5 or 6; and
M is sodium, potassium, or lithium.

2. The method of claim 1 wherein the compound administered has the Formula I 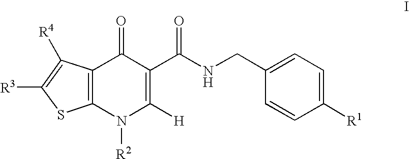
or a pharmaceutically acceptable salt, racemate, solvate, tautomer, optical isomer or prodrug derivative thereof, wherein,
R¹ is
(a) Cl,
(b) Br,
(c) CN,
(d) NO₂, or
(e) F;
R² is
(a) H,
(b) R⁵,
(c) NR⁷R⁸,
(d) SO₂R⁹, or
(e) OR⁹;
R³ is
(a) H,
(b) halo,
(c) aryl,
(d) S(O)ₘR⁶,
(e) (C═O)R⁶,
(f) (C═O)OR⁹,
(g) cyano,
(h) het, wherein said het is bound via a carbon atom,
(i) OR¹⁰,
(j) Ohet,
(k) NR⁷R⁸
(l) SR¹⁰,
(m) Shet,
(n) NHCOR¹²,
(o) NHSO₂R¹², or
(p) C₁₋₇alkyl which may be partially unsaturated and optionally substituted by one or more substituents of the group R¹¹, OR¹³, SR¹⁰, SR¹³, NR⁷R⁸, halo, (C═O)C₁₋₇alkyl, or SOₘR⁹;
R⁴ is
(a) H,
(b) halo,
(c) C₁₋₄alkyl, or
(d) R⁴ together with R³ form a carbocyclic or het, either of which may be optionally substituted by NR⁷R⁸, by C₁₋₇alkyl which may be optionally substituted by OR¹⁴, or by het, wherein said het is bound via a carbon atom;
R⁵ is
(a) (CH₂CH₂O)ᵢR¹⁰,
(b) het, wherein said het is bound via a carbon atom,
(c) aryl,
(d) C₁₋₇alkyl which may be partially unsaturated and is optionally substituted by one or more substituents selected from a group consisting of NR⁷R⁸, R¹¹, SOₘR⁹, and OC₂₋₄alkyl which may be further substituted by het, OR¹⁰, or NR⁷R⁸, or
(e) C₃₋₈cycloalkyl which may be partially unsaturated and optionally substituted by one or more substituents selected from a group consisting of R¹¹, NR⁷R⁸, SOₘR⁹, and C₁₋₇alkyl optionally substituted by R¹¹, NR⁷R⁸, or SOₘR⁹;
R⁶ is
(a) C₁₋₇alkyl,
(b) NR⁷R⁸,
(c) aryl, or
(d) het, wherein said het is bound via a carbon atom;
R⁷ and R⁸ are independently
(a) H,
(b) aryl,
(c) C₁₋₇alkyl which may be partially unsaturated and is optionally substituted by one or more substituents selected from a group consisting of NR¹⁰R¹⁰, R¹¹, SOₘR⁹, CONR¹⁰R¹⁰, or halo, or,
(d) R⁷ and R⁸ together with the nitrogen to which they are attached form a het;
R⁹ is
(a) aryl,
(b) het,
(c) C₃₋₈cycloalkyl, or
(d) C₁₋₇alkyl which may be partially unsaturated and is optionally substituted by one or more substituents selected from a group consisting of NR¹⁰R¹⁰, R¹¹, SH, CONR¹⁰R¹⁰, or halo;
R¹⁰ is
(a) H, or
(b) C₁₋₇alkyl optionally substituted by OH;
R¹¹ is
(a) OR¹⁰,
(b) Ohet,
(c) Oaryl,
(d) CO₂R¹⁰,
(e) het,
(f) aryl, or
(g) CN;
R¹² is
(a) H,
(b) het,
(c) aryl,
(d) C₃₋₈cycloalkyl, or
(e) C₁₋₇alkyl optionally substituted by NR⁷R⁸ or R¹¹;
R¹³ is
(a) (P═O)(OR¹⁴⁾₂,
(b) CO(CH₂)ₙCON(CH₃)—(CH₂)ₙSO₃⁻M⁺,
(c) an amino acid,
(d) C(═O)aryl, or
(e) C(═O)C₁₋₇alkyl optionally substituted by NR⁷R⁸, aryl, het, CO₂H, or O(CH₂)ₙCO₂R¹⁴);
R¹⁴ is
(a) H, or
(b) C₁₋₇alkyl;
each i is independently 2, 3, or 4;
each n is independently 1, 2, 3, 4 or 5;
each m is independently 0, 1, or 2; and
M is sodium, potassium, or lithium;
wherein any aryl is optionally substituted with one or more substituents selected from the group consisting of halo, OH, cyano, CO₂R¹⁴, CF₃, C₁₋₆alkoxy, and C₁₋₆ alkyl which maybe further substituted by one to three SR¹⁴, NR¹⁴R¹⁴, OR¹⁴, het, and CO₂R¹⁴; and
wherein any het is optionally substituted with one or more substituents selected from the group consisting of halo, OH, cyano, phenyl, CO₂R¹⁴, CF₃, C₁₋₆alkoxy, oxo, oxime, and C₁₋₆ alkyl which maybe further substituted by one to three SR¹⁴, NR¹⁴R¹⁴, OR¹⁴, and CO₂R¹⁴;
aryl denotes a phenyl radical or an ortho-fused bicyclic carbocyclic radical having about nine to ten ring atoms in which at least one ring is aromatic. Het is a four-(4), five-(5), six-(6), or seven-(7) membered saturated or unsaturated heterocyclic ring having 1, 2, 3, or 4 heteroatoms selected from the group consisting of oxy, thio, sulfinyl, sulfonyl, and nitrogen, which is optionally fused to a benzene ring, or any bicyclic heterocycle group;
het includes “heteroaryl,” which encompasses a radical attached via a ring carbon of a monocyclic aromatic ring containing five or six ring atoms consisting of carbon and 1, 2, 3, or 4 heteroatoms each selected from the group consisting of non-peroxide oxy, thio, and N(X) wherein X is absent or is H, O, C₁₋₄alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

3. The method of claim 2, wherein R¹ is F, Cl or Br.

4. The method of claim 2, wherein the compound administered is:
(1) N-(4-Chlorobenzyl)-4-hydroxythieno[2,3-b]pyridine-5-carboxamide;
(2) N-(4-Chlorobenzyl)-4-hydroxy-2-iodothieno[2,3-b]pyridine-5-carboxamide;
(3) N-(4-Chlorobenzyl)-4-hydroxy-2-(4-morpholinylsulfonyl)thieno[2,3-b]-pyridine-5-carboxamide;
(4) 2-Bromo-N-(4-chlorobenzyl)-4-hydroxythieno[2,3-b]pyridine-5-carboxamide;
(5) N-(4-Chlorobenzyl)-4-hydroxy-2-(3-hydroxy-1-propynyl)thieno[2,3-b]-pyridine-5-carboxamide;
(6) N-(4-Chlorobenzyl)-4-hydroxy-2-(3-methoxy-1-propynyl)thieno[2,3-b]-pyridine-5-carboxamide;
(7) N-(4-Chlorobenzyl)-4-hydroxy-2-(4-hydroxy-1-butynyl)thieno[2,3-b]-pyridine-5-carboxamide;
(8) N-(4-Chlorobenzyl)-4-hydroxy-2-(3-hydroxypropyl)thieno[2,3-b]pyridine-5-carboxamide;
(9) N-(4-Chlorobenzyl)-2-cyano-4-hydroxythieno[2,3-b]pyridine-5-carboxamide;
(10) Dimethyl 2-[3-(5-{[(4-chlorobenzyl)amino]carbonyl}-4-hydroxythieno[2,3-b]-pyridin-2-yl)-2-propynyl]malonate;
(11) 2-Bromo-N-(4-chlorobenzyl)-7-ethyl-4-oxo-4,7-dihydrothieno[2,3-b]-pyridine-5-carboxamide;
(12) N-(4-Chlorobenzyl)-7-ethyl-4-oxo-4,7-dihydrothieno[2,3-b]pyridine-5-carboxamide;
(13) N-(4-Chlorobenzyl)-7-ethyl-2-iodo-4-oxo-4,7-dihydrothieno[2,3-b]-pyridine-5-carboxamide;
(14) N-(4-Chlorobenzyl)-7-ethyl-2-(3-hydroxy-1-propynyl)-4-oxo-4,7-dihydrothieno[2,3-b]pyridine-5-carboxamide;
(15) N-(4-Chlorobenzyl)-7-ethyl-2-(4-hydroxy-1-butynyl)-4-oxo-4,7-dihydrothieno[2,3-b]pyridine-5-carboxamide;
(16) N-(4-Chlorobenzyl)-7-ethyl-2-(3-hydroxypropyl)-4-oxo-4,7-dihydrothieno[2,3-b]pyridine-5-carboxamide;
(17) N-(4-Chlorobenzyl)-7-(2-hydroxyethyl)-2-(3-hydroxy-1-propynyl)-4-oxo-4,7-dihydrothieno[2,3-b]pyridine-5-carboxamide;
(18) N-(4-Chlorobenzyl)-7-[2-(diethylamino)ethyl]-2-(3-hydroxy-1-propynyl)-4-oxo-4,7-dihydrothieno[2,3-b]pyridine-5-carboxamide;
(19) 2-[5-{[(4-Chlorobenzyl)amino]carbonyl}-2-(3-hydroxy-1-propynyl)-4-oxothieno[2,3-b]pyridin-7(4H)-yl]acetic acid;
(20) N-(4-Chlorobenzyl)-7-ethyl-2-(4-hydroxybutyl)-4-oxo-4,7-dihydrothieno[2,3-b]pyridine-5-carboxamide;
(21) N-(4-Chlorobenzyl)-7-(2-hydroxyethyl)-2-(3-hydroxypropyl)-4-oxo-4,7-dihydrothieno[2,3-b]pyridine-5-carboxamide;
(22) N-(4-Chlorobenzyl)-7-[2-(diethylamino)ethyl]-2-(3-hydroxypropyl)-4-oxo-4,7-dihydrothieno[2,3-b]pyridine-5-carboxamide;
(23) N-(4-Chlorobenzyl)-2-iodo-7-methyl-4-oxo-4,7-dihydrothieno[2,3-b]-pyridine-5-carboxamide;
(24) N-(4-Chlorobenzyl)-2-(3-hydroxy-1-propynyl)-7-methyl-4-oxo-4,7-dihydrothieno[2,3-b]pyridine-5-carboxamide;
(25) N-(4-Chlorobenzyl)-2-(3-hydroxypropyl)-7-methyl-4-oxo-4,7-dihydrothieno[2,3-b]pyridine-5-carboxamide;
(26) N-(4-Chlorobenzyl)-2-iodo-7-isopropyl-4-oxo-4,7-dihydrothieno[2,3-b]-pyridine-5-carboxamide;
(27) N-(4-Chlorobenzyl)-2-(3-hydroxy-1-propynyl)-7-isopropyl-4-oxo-4,7-dihydrothieno[2,3-b]pyridine-5-carboxamide;
(28) N-(4-Chlorobenzyl)-2-(3-hydroxypropyl)-7-isopropyl-4-oxo-4,7-dihydrothieno[2,3-b]pyridine-5-carboxamide;
(29) 4-{[3-(5-{[(4-Chlorobenzyl)amino]carbonyl}-7-ethyl-4-oxo-4,7-dihydrothieno[2,3-b]pyridin-2-yl)-2-propynyl]oxy}-4-oxobutanoic acid;
(30) 3-(5-{[(4-Chlorobenzyl)amino]carbonyl}-7-ethyl-4-oxo-4,7-dihydrothieno[2,3-b]pyridin-2-yl)-2-propynyl 2-(4-morpholinyl)acetate;
(31) 3-(5-{[(4-Chlorobenzyl)amino]carbonyl}-7-ethyl-4-oxo-4,7-dihydrothieno[2,3-b]pyridin-2-yl)-2-propynyl 2-amino-3-methylbutanoate;
(32) 3-(5-{[(4-Chlorobenzyl)amino]carbonyl}-7-ethyl-4-oxo-4,7-dihydrothieno[2,3-b]pyridin-2-yl)-2-propynyl 3-(4-morpholinylmethyl)benzoate;
(33) Methyl-5-{[4-chlorobenzyl)amino]carbonyl}-4-hydroxythienol[2,3-b]pyridine-2-carboxylate;
(34) N-(4-Chlorobenzyl)-4-hydroxy-2-(hydroxymethyl)thieno[2,3-b]pyridine-5-carboxamide;
(35) N-(4-chlorobenzyl)-2-(hydroxymethy)-7-methyl-4-oxo-4,7-dihydrothienol[2,3-b]pyridine-5-carboxamide;
(36) N-(4-chlorobenzyl)-7-methyl-2-(4-morpholinylmethyl)-4-oxo-4,7-dihydrothienol[2,3-b]pyridine-5-carboxamide;
(37) N-(4-chlorobenzyl)-7-methyl-2-(4-morpholinylmethyl)-4-oxo-4,7-dihydrothieno[2,3-b]pyridine-5-carboxamide;
(38) N-(4-chlorobenzyl)-7-methyl-4-oxo-2-(4-thiomorpholinylmethyl)-4,7-dihydrothieno[2,3-b]pyridine-5-carboxamide;
(39) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b] pyridine-5-carboxamide;
(40) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;
(41) N-(4-chlorobenzyl)-4-hydroxy-2-(4-morpholinylmethyl)thieno[2,3-b]pyridine-5-carboxamide;
(42) N-(4-Chlorobenzyl)-7-ethyl-2-(4-morpholinylmethyl)-4-oxo-4,7-dihydrothieno[2,3-b]pyridine-5-carboxamide;
(43) N-(4-Chlorobenzyl)-2-(4-morpholinylmethyl)-4-oxo-7-propyl-4,7-dihydrothieno[2,3-b]pyridine-5-carboxamide;
(44) N-(4-Chlorobenzyl)-7-isopropyl-2-(4-morpholinylmethyl)-4-oxo-4,7-dihydrothieno[2,3-b]pyridine-5-carboxamide;
(45) N-(4-Fluorobenzyl)-7-methyl-2-(4-morpholinylmethyl)-4-oxo-4,7-dihydrothieno[2,3-b]pyridine-5-carboxamide;
(46) N-(4-bromobenzyl)-7-methyl-2-(4-morpholinylmethyl)-4-oxo-4,7-dihydrothieno[2,3-b]pyridine-5-carboxamide;
(47) N-(4-chlorobenzyl)-4-hydroxy-2-(4-morpholinylcarbonyl)thieno[2,3-b]pyridine-5-carboxamide;
(48) N-(4-chlorobenzyl)-7-methyl-2-(4-morpholinylcarbonyl)-4-oxo-4,7-dihydrothieno[2,3-b]pyridine-5-carboxamide;
(49) 7-Benzyl-N-(4-chlorobenzyl)-2-(4-morpholinylmethyl)-4-oxo-4,7-dihydrothieno[2,3-b]pyridine-5-carboxamide;
(50) N-(4-Chlorobenzyl)-7-(3-fluorobenzyl)-2-(4-morpholinylmethyl)-4-oxo-4,7-dihydrothieno[2,3-b]pyridine-5-carboxamide;
(51) N-(4-Chlorobenzyl)-2-(4-morpholinylmethyl)-4-oxo-7-(3-phenylpropyl)-4,7-dihydrothieno[2,3-b]pyridine-5-carboxamide;
(52) N-(4-Chlorobenzyl)-2-(4-morpholinylmethyl)-4-oxo-7-(tetrahydro-2-furanylmethyl)-4,7-dihydrothieno[2,3-b]pyridine-5-carboxamide;
(53) N-(4-Chlorobenzyl)-2-(4-morpholinylmethyl)-4-oxo-7-[2-(1-pyrrolidinyl)ethyl]-4,7-dihydrothieno[2,3-b]pyridine-5-carboxamide;
(54) N-(4-Chlorobenzyl)-2-(4-morpholinylmethyl)-4-oxo-7-(3-pyridinylmethyl)-4,7-dihydrothieno[2,3-b]pyridine-5-carboxamide;
(55) N-(4-Chlorobenzyl)-2-(4-morpholinylmethyl)-4-oxo-7-(4-pyridinylmethyl)-4,7-dihydrothieno[2,3-b]pyridine-5-carboxamide; or
a pharmaceutically acceptable salt thereof.

5. The method of claim 2, wherein the compound administered is:
(1) N-(4-Chlorobenzyl)-7-ethyl-2-(3-hydroxy-1-propynyl)-4-oxo-4,7-dihydro-thieno[2,3-b]pyridine-5-carboxamide;
(2) N-(4-Chlorobenzyl)-7-ethyl-2-(4-hydroxy-1-butynyl)-4-oxo-4,7-dihydro-thieno[2,3-b]pyridine-5-carboxamide;
(3) N-(4-Chlorobenzyl)-7-ethyl-2-(3-hydroxypropyl)-4-oxo-4,7-dihydro-thieno[2,3-b]pyridine-5-carboxamide;
(4) N-(4-Chlorobenzyl)-7-(2-hydroxyethyl)-2-(3-hydroxy-1-propynyl)-4-oxo-4,7-dihydrothieno[2,3-b]pyridine-5-carboxamide;
(5) N-(4-Chlorobenzyl)-7-[2-(diethylamino)ethyl]-2-(3-hydroxy-1-propynyl)-4-oxo-4,7-dihydrothieno[2,3-b]pyridine-5-carboxamide;
(6) N-(4-Chlorobenzyl)-7-ethyl-2-(4-hydroxybutyl)-4-oxo-4,7-dihydro-thieno[2,3-b]pyridine-5-carboxamide;
(7) N-(4-Chlorobenzyl)-7-(2-hydroxyethyl)-2-(3-hydroxypropyl)-4-oxo-4,7-dihydrothieno[2,3-b]pyridine-5-carboxamide;
(8) N-(4-Chlorobenzyl)-7-[2-(diethylamino)ethyl]-2-(3-hydroxypropyl)-4-oxo-4,7-dihydrothieno[2,3-b]pyridine-5-carboxamide;
(9) N-(4-Chlorobenzyl)-2-iodo-7-methyl-4-oxo-4,7-dihydrothieno[2,3-b]-pyridine-5-carboxamide;
(10) N-(4-Chlorobenzyl)-2-(3-hydroxy-1-propynyl)-7-methyl-4-oxo-4,7-dihydrothieno[2,3-b]pyridine-5-carboxamide;
(11) N-(4-Chlorobenzyl)-2-(3-hydroxypropyl)-7-methyl-4-oxo-4,7-dihydro-thieno[2,3-b]pyridine-5-carboxamide;
(12) N-(4-Chlorobenzyl)-2-iodo-7-isopropyl-4-oxo-4,7-dihydrothieno[2,3-b]-pyridine-5-carboxamide;
(13) N-(4-Chlorobenzyl)-2-(3-hydroxy-1-propynyl)-7-isopropyl-4-oxo-4,7-dihydrothieno[2,3-b]pyridine-5-carboxamide;
(14) N-(4-Chlorobenzyl)-2-(3-hydroxypropyl)-7-isopropyl-4-oxo-4,7-dihydro-thieno[2,3-b]pyridine-5-carboxamide;
(15) 4-{[3-(5-{[(4-Chlorobenzyl)amino]carbonyl}-7-ethyl-4-oxo-4,7-dihydro-thieno[2,3-b]pyridin-2-yl)-2-propynyl]oxy}-4-oxobutanoic acid;
(16) 3-(5-{[(4-Chlorobenzyl)amino]carbonyl}-7-ethyl-4-oxo-4,7-dihydro-thieno[2,3-b]pyridin-2-yl)-2-propynyl 2-(4-morpholinyl)acetate;
(17) 3-(5-{[(4-Chlorobenzyl)amino]carbonyl}-7-ethyl-4-oxo-4,7-dihydro-thieno[2,3-b]pyridin-2-yl)-2-propynyl 2-amino-3-methylbutanoate;
(18) 3-(5-{[(4-Chlorobenzyl)amino]carbonyl}-7-ethyl-4-oxo-4,7-dihydro-thieno[2,3-b]pyridin-2-yl)-2-propynyl 3-(4-morpholinylmethyl)benzoate;
(19) N-(4-chlorobenzyl)-7-methyl-2-(4-morpholinylmethyl)-4-oxo-4,7-dihydrothienol[2,3-b]pyridine-5-carboxamide;
(20) N-(4-chlorobenzyl)-7-methyl-2-(4-morpholinylmethyl)-4-oxo-4,7-dihydrothieno[2,3-b]pyridine-5-carboxamide;
(21) N-(4-chlorobenzyl)-7-methyl-4-oxo-2-(4-thiomorpholinylmethyl)-4,7-dihydrothieno[2,3-b]pyridine-5-carboxamide;
(22) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b]pyridine-5-carboxamide;
(23) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;
(24) N-(4-Chlorobenzyl)-7-ethyl-2-(4-morpholinylmethyl)-4-oxo-4,7-dihydrothieno[2,3-b]pyridine-5-carboxamide;
(25) N-(4-Chlorobenzyl)-2-(4-morpholinylmethyl)-4-oxo-7-propyl-4,7-dihydrothieno[2,3-b]pyridine-5-carboxamide;
(26) N-(4-Chlorobenzyl)-7-isopropyl-2-(4-morpholinylmethyl)-4-oxo-4,7-dihydrothieno[2,3-b]pyridine-5-carboxamide;
(27) N-(4-Fluorobenzyl)-7-methyl-2-(4-morpholinylmethyl)-4-oxo-4,7-dihydrothieno[2,3-b]pyridine-5-carboxamide;
(28) N-(4-bromobenzyl)-7-methyl-2-(4-morpholinylmethyl)-4-oxo-4,7-dihydrothieno[2,3-b]pyridine-5-carboxamide;
(29) 7-Benzyl-N-(4-chlorobenzyl)-2-(4-morpholinylmethyl)-4-oxo-4,7-dihydrothieno[2,3-b]pyridine-5-carboxamide;
(30) N-(4-Chlorobenzyl)-7-(3-fluorobenzyl)-2-(4-morpholinylmethyl)-4-oxo-4,7-dihydrothieno[2,3-b]pyridine-5-carboxamide;
(31) N-(4-Chlorobenzyl)-2-(4-morpholinylmethyl)-4-oxo-7-(3-phenylpropyl)-4,7-dihydrothieno[2,3-b]pyridine-5-carboxamide;
(32) N-(4-Chlorobenzyl)-2-(4-morpholinylmethyl)-4-oxo-7-(tetrahydro-2-furanylmethyl)-4,7-dihydrothieno[2,3-b]pyridine-5-carboxamide;
(33) N-(4-Chlorobenzyl)-2-(4-morpholinylmethyl)-4-oxo-7-[2-(1-pyrrolidinyl)ethyl]-4,7-dihydrothieno[2,3-b]pyridine-5-carboxamide;
(34) N-(4-Chlorobenzyl)-2-(4-morpholinylmethyl)-4-oxo-7-(3-pyridinylmethyl)-4,7-dihydrothieno[2,3-b]pyridine-5-carboxamide;
(35) N-(4-Chlorobenzyl)-2-(4-morpholinylmethyl)-4-oxo-7-(4-pyridinylmethyl)-4,7-dihydrothieno[2,3-b]pyridine-5-carboxamide; or
a pharmaceutically acceptable salt thereof.

6. The method of claim 2, wherein the compound administered is:
(1) N-(4-Chlorobenzyl)-7-ethyl-2-(3-hydroxy-1-propynyl)-4-oxo-4,7-dihydro-thieno[2,3-b]pyridine-5-carboxamide;
(2) N-(4-Chlorobenzyl)-7-ethyl-2-(4-hydroxy-1-butynyl)-4-oxo-4,7-dihydro-thieno[2,3-b]pyridine-5-carboxamide;
(3) N-(4-Chlorobenzyl)-7-ethyl-2-(3-hydroxypropyl)-4-oxo-4,7-dihydro-thieno[2,3-b]pyridine-5-carboxamide;
(4) N-(4-Chlorobenzyl)-7-(2-hydroxyethyl)-2-(3-hydroxy-1-propynyl)-4-oxo-4,7-dihydrothieno[2,3-b]pyridine-5-carboxamide;
(5) N-(4-Chlorobenzyl)-7-[2-(diethylamino)ethyl]-2-(3-hydroxy-1-propynyl)-4-oxo-4,7-dihydrothieno[2,3-b]pyridine-5-carboxamide;
(6) N-(4-Chlorobenzyl)-7-ethyl-2-(4-hydroxybutyl)-4-oxo-4,7-dihydro-thieno[2,3-b]pyridine-5-carboxamide;
(7) N-(4-Chlorobenzyl)-7-(2-hydroxyethyl)-2-(3-hydroxypropyl)-4-oxo-4,7-dihydrothieno[2,3-b]pyridine-5-carboxamide;
(8) N-(4-Chlorobenzyl)-7-[2-(diethylamino)ethyl]-2-(3-hydroxypropyl)-4-oxo-4,7-dihydrothieno[2,3-b]pyridine-5-carboxamide;
(9) N-(4-Chlorobenzyl)-2-iodo-7-methyl-4-oxo-4,7-dihydrothieno[2,3-b]-pyridine-5-carboxamide;
(10) N-(4-Chlorobenzyl)-2-(3-hydroxy-1-propynyl)-7-methyl-4-oxo-4,7-dihydrothieno[2,3-b]pyridine-5-carboxamide;
(11) N-(4-Chlorobenzyl)-2-(3-hydroxypropyl)-7-methyl-4-oxo-4,7-dihydro-thieno[2,3-b]pyridine-5-carboxamide;
(12) N-(4-Chlorobenzyl)-2-iodo-7-isopropyl-4-oxo-4,7-dihydrothieno[2,3-b]-pyridine-5-carboxamide;
(13) N-(4-Chlorobenzyl)-2-(3-hydroxy-1-propynyl)-7-isopropyl-4-oxo-4,7-dihydrothieno[2,3-b]pyridine-5-carboxamide;
(14) N-(4-Chlorobenzyl)-2-(3-hydroxypropyl)-7-isopropyl-4-oxo-4,7-dihydro-thieno[2,3-b]pyridine-5-carboxamide;
(15) N-(4-chlorobenzyl)-7-methyl-2-(4-morpholinylmethyl)-4-oxo-4,7-dihydrothieno[2,3-b]pyridine-5-carboxamide;
(16) N-(4-chlorobenzyl)-7-methyl-2-(4-morpholinylmethyl)-4-oxo-4,7-dihydrothieno[2,3-b]pyridine-5-carboxamide;
(17) N-(4-chlorobenzyl)-7-methyl-4-oxo-2-(4-thiomorpholinylmethyl)-4,7-dihydrothieno[2,3-b]pyridine-5-carboxamide;
(18) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b]pyridine-5-carboxamide;
(19) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;
(20) N-(4-Chlorobenzyl)-7-ethyl-2-(4-morpholinylmethyl)-4-oxo-4,7-dihydrothieno[2,3-b]pyridine-5-carboxamide;
(21) N-(4-Chlorobenzyl)-2-(4-morpholinylmethyl)-4-oxo-7-propyl-4,7-dihydrothieno[2,3-b]pyridine-5-carboxamide;
(22) N-(4-Chlorobenzyl)-7-isopropyl-2-(4-morpholinylmethyl)-4-oxo-4,7-dihydrothieno[2,3-b]pyridine-5-carboxamide;
(23) N-(4-Fluorobenzyl)-7-methyl-2-(4-morpholinylmethyl)-4-oxo-4,7-dihydrothieno[2,3-b]pyridine-5-carboxamide;
(24) N-(4-bromobenzyl)-7-methyl-2-(4-morpholinylmethyl)-4-oxo-4,7-dihydrothieno[2,3-b]pyridine-5-carboxamide;
(25) 7-Benzyl-N-(4-chlorobenzyl)-2-(4-morpholinylmethyl)-4-oxo-4,7-dihydrothieno[2,3-b]pyridine-5-carboxamide;
(26) N-(4-Chlorobenzyl)-7-(3-fluorobenzyl)-2-(4-morpholinylmethyl)-4-oxo-4,7-dihydrothieno[2,3-b]pyridine-5-carboxamide;
(27) N-(4-Chlorobenzyl)-2-(4-morpholinylmethyl)-4-oxo-7-(tetrahydro-2-furanylmethyl)-4,7-dihydrothieno[2,3-b]pyridine-5-carboxamide;
(28) N-(4-Chlorobenzyl)-2-(4-morpholinylmethyl)-4-oxo-7-[2-(1-pyrrolidinyl)ethyl]-4,7-dihydrothieno[2,3-b]pyridine-5-carboxamide;
(29) N-(4-Chlorobenzyl)-2-(4-morpholinylmethyl)-4-oxo-7-(3-pyridinylmethyl)-4,7-dihydrothieno[2,3-b]pyridine-5-carboxamide;
(30) N-(4-Chlorobenzyl)-2-(4-morpholinylmethyl)-4-oxo-7-(4-pyridinylmethyl)-4,7-dihydrothieno[2,3-b]pyridine-5-carboxamide; or
a pharmaceutically acceptable salt thereof.

7. The method of claim 2, wherein the compound administered is:
(1) N-(4-Chlorobenzyl)-7-[2-(diethylamino)ethyl]-2-(3-hydroxypropyl)-4-oxo-4,7-dihydrothieno[2,3-b]pyridine-5-carboxamide;
(2) N-(4-Chlorobenzyl)-2-(3-hydroxy-1-propynyl)-7-methyl-4-oxo-4,7-dihydro-thieno[2,3-b]pyridine-5-carboxamide;
(3) N-(4-Chlorobenzyl)-2-(3-hydroxypropyl)-7-methyl-4-oxo-4,7-dihydro-thieno[2,3-b]pyridine-5-carboxamide;
(4) N-(4-chlorobenzyl)-7-methyl-2-(4-morpholinylmethyl)-4-oxo-4,7-dihydrothieno[2,3-b]pyridine-5-carboxamide;
(5) N-(4-chlorobenzyl)-2-(((2-hydroxy-2-(4-hydroxyphenyl)ethyl)(methyl)amino)methyl)-7-methyl-4-oxo-4,7-dihydrothieno[2,3-b]pyridine-5-carboxamide;
(6) N-(4-chlorobenzyl)-2-(((2-hydroxy-2-phenylethyl)(methyl)amino)methyl)-7-methyl-4-oxo-4,7-dihydrothieno[2,3-b]pyridine-5-carboxamide;
(7) N-(4-Chlorobenzyl)-7-ethyl-2-(4-morpholinylmethyl)-4-oxo-4,7-dihydrothieno[2,3-b]pyridine-5-carboxamide;
(8) N-(4-Chlorobenzyl)-2-(4-morpholinylmethyl)-4-oxo-7-propyl-4,7-dihydrothieno[2,3-b]pyridine-5-carboxamide; or
a pharmaceutically acceptable salt thereof.

8. The method of claim 2, wherein the compound administered is N(4-chlorobenzyl)-7-methyl-2-(4-morpholinylmethyl)-4-oxo-4,7-dihydrothieno[2,3-b]pyridine-5-carboxamide or a pharmaceutically acceptable salt thereof.

9. The method of claim 1, wherein the compound administered has the Formula I′ 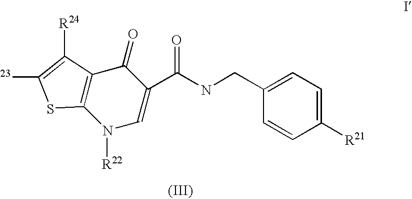
or a pharmaceutically acceptable salt, racemate, solvate, tautomer, optical isomer or prodrug derivative thereof wherein,
R²¹ is Cl, Br, CN, or NO₂;
R is H, —(CH₂CH₂O)ₙH, —(CH₂CH₂O)ₙCH₃, SO₂R³⁵ or COR³⁵, C₁₋₇alkyl which may be partially unsaturated and optionally substituted by R³⁶, C₂₋₇alkyl which may be partially unsaturated and optionally substituted by R³³, or C₃₋₈cycloalkyl which may be partially unsaturated and optionally substituted by R³⁶, R³³ or R³⁴;
each R²³ and R²⁴ is independently H, halo, aryl, S(O)ₘR³⁰, COR³⁰, cyano, het, CF₃, OR²⁹, OR³¹, SR²⁹, SR³¹, NR²⁵R²⁶, CH(OR²⁹)R²⁷, CO₂R²⁹, CH(CO₂R²⁹)₂, NHCOR²⁷, or NHS(O)₂R²⁷ or C₁₋₇alkyl which may be partially unsaturated and optionally substituted by R²⁸;
each R²⁵ and R²⁶ is independently H or C₁₋₇alkyl;
R²⁷ is C₁₋₇alkyl optionally substituted by R³⁶ or C₂₋₇alkyl optionally substituted by R³³;
R²⁸ is cyano, halo, CF₃, aryl, het, C(═O)C₁₋₇ alkyl, CO₂C₁₋₇alkyl, OR²⁹, OR³¹, OR³², SR²⁹, SR³¹, SR³², NR²⁵R²⁶, CH(OR²⁹)R²⁷, CO₂R²⁹ or CH(CO₂R²⁹)₂;
R²⁹ is H or C₁₋₇alkyl;
R³⁰ is C₁₋₇alkyl, NR²⁵R²⁶, aryl or het;
R³¹ is C₂₋₇alkyl substituted by OH;
R³² is (P═O)(OR²⁹)₂, CO(CH₂)ₙCON(CH₃)—(CH₂)ₙSO₃⁻M⁺, an amino acid, C(═O)aryl, or C(═O)C₁₋₇alkyl optionally substituted by NR²⁵R²⁶, aryl, het, carboxy, or O(CH₂)ₙCO₂R²⁹;
R³³ is hydroxy or NR²⁵R²⁶;
R³⁴ is C₁₋₇alkyl optionally substituted R³³;
R³⁵ is C₁₋₇alkyl, aryl or het;
R³⁶ is CO₂H or CO₂C₁₋₇alkyl
each n is independently 1, 2, 3, 4, or 5;
each m is independently 0, 1, or 2;
M is a pharmaceutically acceptable cation (e.g. sodium, potassium, or lithium);
wherein any aryl, or het is optionally substituted with one or more substituents (e.g. 1, 2, 3, 4, or 5) independently selected from the group consisting of halo, cyano, trifluoromethyl, trifluoromethoxy, hydroxy, carboxy, OR²⁷, phenyl, phenoxy, (C₁₋₇alkoxy)carbonyl, SR³¹, and C₁₋₇ alkyl optionally substituted with one or more substituents independently selected from the group consisting of cyano, aryl, mercapto, het, R³⁶, OR²⁷, SR²⁷, and SR³¹; wherein phenyl or phenoxy is optionally substituted with one or more substituents independently selected from cyano, halo, trifluoromethyl, trifluoromethoxy, carboxy, het, OR³¹, and R²⁷.

10. A method of claim 9, wherein a specific value for R²¹ is Cl.

11. A method of claim 9, wherein a specific value for R²¹ is CN, or NO₂.

12. A method of claim 9, with the proviso that when R²¹ is Cl and R²² is H, R²³ is other than CH₂OH.

13. A method of claim 9, wherein the compound administered has the formula I or I′ and wherein any aryl, or het is optionally substituted with one or two substituents selected from the group consisting of halo, cyano, het trifluoromethyl, trifluoromethoxy, hydroxy C₁₋₇alkoxy, and C₁₋₇alkyl; or a pharmaceutically acceptable salt thereof.

14. The method of claim 1, wherein the compound administered has the Formula II 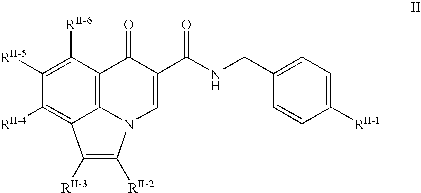
or a pharmaceutically acceptable salt, racemate, solvate, tautomer, optical isomer or prodrug derivative thereof wherein:
R^{II-1}is
(a) F,
(b) Cl,
(c) Br,
(d) CN or
(e) NO₂;
R^{II-2} and R^{II-3} are independently
(a) H,
(b) halo,
(c) OR^{II-11},
(d) C(═O)R^{II-7},
(e) C(═O)OR^{II-11},
(f) C₃₋₈cycloalkyl, or
(g) C₁₋₇alkyl which may be partially unsaturated and optionally substituted by one or more halo, C₃₋₈cycloalkyl, R^{II-12}, OR^{II-14}, SR^{II-11}, SR^{II-14}, NR^{II-8}R^{II-9}, NR^{II-11}C(O)R^{II-7}, (C═O)C₁₋₇ alkyl, or SOₘR^{II-10};
R^{II-4}, and R^{II-5} are independently
(a) H,
(b) halo,
(c) aryl,
(d) S(O)ₘR^{II-7},
(e) (C═O)R^{II-7},
(f) (C═O)OR^{II-10},
(g) CN,
(h) het, wherein said het is bound via a carbon atom,
(i) OR^{II-11},
(j) Ohet,
(k) NR^{II-8}R^{II-9}
(l) SR^{II-11},
(m) Shet,
(n) NHCOR^{II-13},
(o) NHSO₂R^{II-13},
(p) C₃₋₈cycloalkyl, or
(q) C₁₋₇alkyl which may be partially unsaturated and optionally substituted by one or more R^{II-12}, OR^{II-14}, SR^{II-11}, SR^{II-14}, NR^{II-8}R^{II-9}, halo, C₃₋₈cycloalkyl, (C═O)C₁₋₇alkyl, or SOₘR^{II-10};
R^{II-6} is
(a) H,
(b) halo,
(c) C₃₋₈cycloalkyl, or
(d) C₁₋₄alkyl optionally substituted by 1-3 halo;
R^{II-7} is
(a) C₁₋₇alkyl,
(b) C₃₋₈cycloalkyl,
(c) NR^{II-8}R^{II-9},
(d) aryl, or
(e) het, wherein said het is bonded via a carbon atom;
R^{II-8} and R^{II-9} are independently
(a) H,
(b) aryl,
(c) C₃₋₈cycloalkyl,
(d) C₁₋₇alkyl which may be partially unsaturated and is optionally substituted by one or more NR^{II-11}R^{II-1}, R^{II-12}, SOₘR^{II-10}, CONR^{II-11}R^{II-11}, OH, aryl, het, C₃₋₈cycloalkyl, or halo, or
(e) R^{II-8} and R^{II-9} together with the nitrogen to which they are attached for a het;
R^{II-10} is
(a) aryl,
(b) het,
(c) C₃₋₈cycloalkyl, or
(e) C₁₋₇alkyl which may be partially unsaturated and is optionally substituted by one or more NR^{II-11}R^{II-11}, R^{II-12}, SH, CONR^{II-11}R^{II-12}, C₃₋₈cycloalkyl, or halo;
R^{II-11} is
(a) H,
(b) aryl,
(c) C₃₋₈cycloalkyl, or
(d) C₁₋₇alkyl optionally substituted by OH;
R^{II-12} is
(a) OR^{II-11},
(b) Ohet,
(c) Oaryl,
(d) CO₂R^{II-11},
(e) het,
(f) aryl, or
(g) CN;
R^{II-13} is
(a) H,
(b) het,
(c) aryl,
(d) C₃₋₈cycloalkyl, or
(e) C₁₋₇alkyl optionally substituted by NR^{II-11}R^{II-11} or R^{II-12};
R^{II-14} is
(a) (P═O)(OR^{II-15})₂,
(b) CO(CH₂)ₙCON(CH₃)—(CH₂)ₙSO₃⁻M⁺,
(c) an amino acid,
(d) C(═O)aryl, or
(e) C(═O)C₁₋₇alkyl optionally substituted by NR^{II-11}R^{II-11}, aryl, het, CO₂H, or O(CH₂)ₙCO₂R^{II-15};
R^{II-15} is
(a) H, or
(b) C₁₋₇alkyl;
aryl in Formula II is a phenyl radical or an ortho-fused bicyclic carbocyclic radical wherein at least one ring is aromatic; at each occurrence, aryl may be additionally substituted with one or more halo, CN, CO₂R^{II-11}, SR^{II-11}, OR^{II-11}, NR^{II-11}R^{II-11}, C₁₋₄alkyl, CF₃, or C₃₋₈cycloalkyl;
het in Formula II is a four-(4), five-(5), six-(6), or seven-(7) membered saturated or unsaturated heterocyclic ring having 1, 2, or 3 heteroatoms selected from the group consisting of O, SOₘ, and NX; wherein X is H, C₁₋₄alkyl or absence, wherein het is optionally fused to a benzene ring, or any bicyclic heterocycle group; at each occurrence, het may be additionally substituted with one or more halo, CN, CO₂R^{II-11}, SR^{II-11}, OR^{II-11}, NR^{II-11}R^{II-11}, C(═O)R^{II-13}, C₁₋₄alkyl, CF₃, C₃₋₈cycloalkyl, oxo or oxime;
at each occurrence, m is independently 0, 1, or 2;
at each occurrence, n is independently 1, 2, 3, 4, 5 or 6; and
M is sodium, potassium, or lithium.

15. The method of claim 14, wherein R^{II-1} is Cl.

16. The method of claim 14, wherein the compound administered is
(a) N-(4-chlorobenzyl)-2-(hydroxymethyl)-6-oxo-6H-pyrrolo[3,2,1-ij]quinoline-5-carboxamide;
(b) N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide;
(c) N-(4-chlorobenzyl)-2-(hydroxymethyl)-8-(4-morpholinylmethyl)-6-oxo-6H-pyrrolo[3,2,1-ij]quinoline-5-carboxamide;
(d) N-(4-chlorobenzyl)-2-(2-hydroxyethyl)-8-(4-morpholinylmethyl)-6-oxo-6H-pyrrolo[3,2,1-ij]quinoline-5-carboxamide;
(e) N-(4-chlorobenzyl)-2-(2-morpholin-4-ylethyl)-8-(4-morpholinylmethyl)-6-oxo-6H-pyrrolo[3,2,1-ij]quinoline-5-carboxamide;
(f) N-(4-chlorobenzyl)-2-[2-(diethylamino)ethyl]-8-(4-morpholinylmethyl)-6-oxo-6H-pyrrolo[3,2,1-ij]quinoline-5-carboxamide;
(g) N-(4-chlorobenzyl)-2-[2-(4-methylpiperazin-1-yl)ethyl]-8-(morpholin-4-ylmethyl)-6-oxo-6H-pyrrolo[3,2,1-ij]quinoline-5-carboxamide;
(h) N-(4-chlorobenzyl)-2-[2-(2-ethylpiperidin-1-yl)ethyl]-8-(morpholin-4-ylmethyl)-6-oxo-6H-pyrrolo[3,2,1-ij]quinoline-5-carboxamide;
(i) N-(4-chlorobenzyl)-2-[3-(4-methylpiperazin-1-yl)propyl]-8-(morpholin-4-ylmethyl)-6-oxo-6H-pyrrolo[3,2,1-ij]quinoline-5-carboxamide;
(j) N-(4-chlorobenzyl)-8-(morpholin-4-ylmethyl)-6-oxo-2-(2-piperidin-1-ylethyl)-6H-pyrrolo[3,2,1-ij]quinoline-5-carboxamide;
(k) N-(4-chlorobenzyl)-8-(morpholin-4-ylmethyl)-2-(3-morpholin-4-ylpropyl)-6-oxo-6H-pyrrolo[3,2,1-ij]quinoline-5-carboxamide;
(l) N-(4-chlorobenzyl)-2-[(1,1-dioxido-4-thiomorpholinyl)methyl]-8-(4-morpholinylmethyl)-6-oxo-6H-pyrrolo[3,2,1-ij]quinoline-5-carboxamide;
(m) N-(4-chlorobenzyl)-2-[(1,1-dioxido-4-thiomorpholinyl)methyl]-1-iodo-8-(4-morpholinylmethyl)-6-oxo-6H-pyrrolo[3,2,1-ij]quinoline-5-carboxamide;
(n) N-(4-chlorobenzyl)-2-(3-hydroxypropyl)-8-(4-morpholinylmethyl)-6-oxo-6H-pyrrolo[3,2,1-ij]quinoline-5-carboxamide;
(o) 2-{[(aminocarbonyl)amino]methyl}-N-(4-chlorobenzyl)-8-(4-morpholinylmethyl)-6-oxo-6H-pyrrolo[3,2,1-ij]quinoline-5-carboxamide;
(p) N-(4-chlorobenzyl)-2-[(1R)-1-hydroxyethyl]-8-(4-morpholinylmethyl)-6-oxo-6H-pyrrolo[3,2,1-ij]quinoline-5-carboxamide;
(q) N-(4-chlorobenzyl)-2-(methoxymethyl)-8-(4-morpholinylmethyl)-6-oxo-6H-pyrrolo[3,2,1-ij]quinoline-5-carboxamide;
(r) N-(4-chlorobenzyl)-2-[(ethylsulfanyl)methyl]-8-(4-morpholinylmethyl)-6-oxo-6H-pyrrolo[3,2,1-ij]quinoline-5-carboxamide;
(s) N-(4-chlorobenzyl)-8-(4-morpholinylmethyl)-6-oxo-2-[(phenylsulfanyl)methyl]-6H-pyrrolo[3,2,1-ij]quinoline-5-carboxamide;
(t) N-(4-chlorobenzyl)-2-[(methylamino)methyl]-8-(4-morpholinylmethyl)-6-oxo-6H-pyrrolo[3,2,1-ij]quinoline-5-carboxamide;
(u) N-(4-chlorobenzyl)-2-[(dimethylamino)methyl]-8-(4-morpholinylmethyl)-6-oxo-6H-pyrrolo[3,2,1-ij]quinoline-5-carboxamide;
(v) N-(4-chlorobenzyl)-2-(2-hydroxyethyl)-6-oxo-6H-pyrrolo[3,2,1-ij]quinoline-5-carboxamide; or
(w) N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

17. The method of claim 14, wherein the compound administered is
(a) N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide;
(b) N-(4-chlorobenzyl)-2-(hydroxymethyl)-8-(4-morpholinylmethyl)-6-oxo-6H-pyrrolo[3,2,1-ij]quinoline-5-carboxamide;
(c) N-(4-chlorobenzyl)-2-(2-hydroxyethyl)-8-(4-morpholinylmethyl)-6-oxo-6H-pyrrolo[3,2,1-ij]quinoline-5-carboxamide;
(d) N-(4-chlorobenzyl)-2-(2-morpholin-4-ylethyl)-8-(4-morpholinylmethyl)-6-oxo-6H-pyrrolo[3,2,1-ij]quinoline-5-carboxamide;
(e) N-(4-chlorobenzyl)-2-[(1,1-dioxido-4-thiomorpholinyl)methyl]-8-(4-morpholinylmethyl)-6-oxo-6H-pyrrolo[3,2,1-ij]quinoline-5-carboxamide;
(f) N-(4-chlorobenzyl)-2-[(1,1-dioxido-4-thiomorpholinyl)methyl]-1-iodo-8-(4-morpholinylmethyl)-6-oxo-6H-pyrrolo[3,2,1-ij]quinoline-5-carboxamide;
(g) N-(4-chlorobenzyl)-2-(3-hydroxypropyl)-8-(4-morpholinylmethyl)-6-oxo-6H-pyrrolo[3,2,1-ij]quinoline-5-carboxamide;
(h) 2-{[(aminocarbonyl)amino]methyl}-N-(4-chlorobenzyl)-8-(4-morpholinylmethyl)-6-oxo-6H-pyrrolo[3,2,1-ij]quinoline-5-carboxamide;
(i) N-(4-chlorobenzyl)-2-[(1R)-1-hydroxyethyl]-8-(4-morpholinylmethyl)-6-oxo-6H-pyrrolo[3,2,1-ij]quinoline-5-carboxamide;
(j) N-(4-chlorobenzyl)-2-(methoxymethyl)-8-(4-morpholinylmethyl)-6-oxo-6H-pyrrolo[3,2,1-ij]quinoline-5-carboxamide;
(k) N-(4-chlorobenzyl)-2-[(ethylsulfanyl)methyl]-8-(4-morpholinylmethyl)-6-oxo-6H-pyrrolo[3,2,1-ij]quinoline-5-carboxamide;
(l) N-(4-chlorobenzyl)-8-(4-morpholinylmethyl)-6-oxo-2-[(phenylsulfanyl)methyl]-6H-pyrrolo[3,2,1-ij]quinoline-5-carboxamide;
(m) N-(4-chlorobenzyl)-2-[(methylamino)methyl]-8-(4-morpholinylmethyl)-6-oxo-6H-pyrrolo[3,2,1-ij]quinoline-5-carboxamide;
(n) N-(4-chlorobenzyl)-2-[(dimethylamino)methyl]-8-(4-morpholinylmethyl)-6-oxo-6H-pyrrolo[3,2,1-ij]quinoline-5-carboxamide; or
(o) N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

18. The method of claim 14, wherein the compound is
(gg) N-(4-chlorobenzyl)-2-(hydroxymethyl)-6-oxo-6H-pyrrolo[3,2,1-ij]quinoline-5-carboxamide;
(hh) N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide;
(ii) N-(4-chlorobenzyl)-2-(hydroxymethyl)-8-(4-morpholinylmethyl)-6-oxo-6H-pyrrolo[3,2,1-ij]quinoline-5-carboxamide;
(jj) N-(4-chlorobenzyl)-2-(2-hydroxyethyl)-8-(4-morpholinylmethyl)-6-oxo-6H-pyrrolo[3,2,1-ij]quinoline-5-carboxamide;
(kk) N-(4-chlorobenzyl)-2-(2-morpholin-4-ylethyl)-8-(4-morpholinylmethyl)-6-oxo-6H-pyrrolo[3,2,1-ij]quinoline-5-carboxamide;
(ll) N-(4-chlorobenzyl)-2-[2-(diethylamino)ethyl]-8-(4-morpholinylmethyl)-6-oxo-6H-pyrrolo[3,2,1-ij]quinoline-5-carboxamide;
(mm) N-(4-chlorobenzyl)-2-[2-(4-methylpiperazin-1-yl)ethyl]-8-(morpholin-4-ylmethyl)-6-oxo-6H-pyrrolo[3,2,1-ij]quinoline-5-carboxamide;
(nn) N-(4-chlorobenzyl)-2-[2-(2-ethylpiperidin-1-yl)ethyl]-8-(morpholin-4-ylmethyl)-6-oxo-6H-pyrrolo[3,2,1-ij]quinoline-5-carboxamide;
(oo) N-(4-chlorobenzyl)-2-[3-(4-methylpiperazin-1-yl)propyl]-8-(morpholin-4-ylmethyl)-6-oxo-6H-pyrrolo[3,2,1-ij]quinoline-5-carboxamide;
(pp) N-(4-chlorobenzyl)-8-(morpholin-4-ylmethyl)-6-oxo-2-(2-piperidin-1-ylethyl)-6H-pyrrolo[3,2,1-ij]quinoline-5-carboxamide;
(qq) N-(4-chlorobenzyl)-8-(morpholin-4-ylmethyl)-2-(3-morpholin-4-ylpropyl)-6-oxo-6H-pyrrolo[3,2,1-ij]quinoline-5-carboxamide;
(rr) N-(4-chlorobenzyl)-2-[(1,1-dioxido-4-thiomorpholinyl)methyl]-8-(4-morpholinylmethyl)-6-oxo-6H-pyrrolo[3,2,1-ij]quinoline-5-carboxamide;
(ss) N-(4-chlorobenzyl)-2-[(1,1-dioxido-4-thiomorpholinyl)methyl]-1-iodo-8-(4-morpholinylmethyl)-6-oxo-6H-pyrrolo[3,2,1-ij]quinoline-5-carboxamide;
(tt) N-(4-chlorobenzyl)-2-(3-hydroxypropyl)-8-(4-morpholinylmethyl)-6-oxo-6H-pyrrolo[3,2,1-ij]quinoline-5-carboxamide;
(uu) 2-{[(aminocarbonyl)amino]methyl}-N-(4-chlorobenzyl)-8-(4-morpholinylmethyl)-6-oxo-6H-pyrrolo[3,2,1-ij]quinoline-5-carboxamide;
(vv) N-(4-chlorobenzyl)-2-[(1R)-1-hydroxyethyl]-8-(4-morpholinylmethyl)-6-oxo-6H-pyrrolo[3,2,1-ij]quinoline-5-carboxamide;
(ww) N-(4-chlorobenzyl)-2-(methoxymethyl)-8-(4-morpholinylmethyl)-6-oxo-6H-pyrrolo[3,2,1-ij]quinoline-5-carboxamide;
(xx) N-(4-chlorobenzyl)-2-[(ethylsulfanyl)methyl]-8-(4-morpholinylmethyl)-6-oxo-6H-pyrrolo[3,2,1-ij]quinoline-5-carboxamide;
(yy) N-(4-chlorobenzyl)-8-(4-morpholinylmethyl)-6-oxo-2-[(phenylsulfanyl)methyl]-6H-pyrrolo[3,2,1-ij]quinoline-5-carboxamide;
(zz) N-(4-chlorobenzyl)-2-[(methylamino)methyl]-8-(4-morpholinylmethyl)-6-oxo-6H-pyrrolo[3,2,1-ij]quinoline-5-carboxamide;
(aaa) N-(4-chlorobenzyl)-2-[(dimethylamino)methyl]-8-(4-morpholinylmethyl)-6-oxo-6H-pyrrolo[3,2,1-ij]quinoline-5-carboxamide;
(bbb) N-(4-chlorobenzyl)-2-(2-hydroxyethyl)-6-oxo-6H-pyrrolo[3,2,1-ij]quinoline-5-carboxamide;
(ccc) N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide;
(ddd) N-(4-chlorobenzyl)-2-[2-hydroxy-1-(hydroxymethyl)ethyl]-8-(4-morpholinylmethyl)-6-oxo-6H-pyrrolo[3,2,1-ij]quinoline-5-carboxamide;
(eee) N-(4-chlorobenzyl)-2-[1,2-dihydroxy-1-(hydroxymethyl)ethyl]-8-(4-morpholinylmethyl)-6-oxo-6H-pyrrolo[3,2,1-ij]quinoline-5-carboxamide;
(fff) N-(4-chlorobenzyl)-2-[(ethylsulfonyl)methyl]-8-(4-morpholinylmethyl)-6-oxo-6H-pyrrolo[3,2,1-ij]quinoline-5-carboxamide;
(ggg) N-(4-chlorobenzyl)-2-[(ethylsulfinyl)methyl]-8-(4-morpholinylmethyl)-6-oxo-6H-pyrrolo[3,2,1-ij]quinoline-5-carboxamide;
(hhh) 2-{[bis(2-hydroxyethyl)amino]methyl}-N-(4-chlorobenzyl)-8-(4-morpholinylmethyl)-6-oxo-6H-pyrrolo[3,2,1-ij]quinoline-5-carboxamide;
(iii) N-(4-chlorobenzyl)-2-[(2-hydroxyethoxy)methyl]-8-(4-morpholinylmethyl)-6-oxo-6H-pyrrolo[3,2,1-ij]quinoline-5-carboxamide;
(jjj) N-(4-chlorobenzyl)-2-(1,2-dihydroxyethyl)-8-(4-morpholinylmethyl)-6-oxo-6H-pyrrolo[3,2,1-ij]quinoline-5-carboxamide;
(kkk) N-(4-chlorobenzyl)-8-(4-morpholinylmethyl)-6-oxo-2-(1,2,3-trihydroxypropyl)6H-pyrrolo[3,2,1-ij]quinoline-5-carboxamide;
(lll) N-(4-chlorobenzyl)-2-[3-hydroxy-2-(hydroxymethyl)propyl]-8-(4-morpholinylmethyl)-6-oxo-6H-pyrrolo[3,2,1-ij]quinoline-5-carboxamide;
(mmm) N-(4-chlorobenzyl)-1-(hydroxymethyl)-8-(morpholin-4-ylmethyl)-6-oxo-6H-pyrrolo[3,2,1-ij]quinoline-5-carboxamide;
(nnn) N-(4-chlorobenzyl)-1-(2-hydroxyethyl)-8-(morpholin-4-ylmethyl)-6-oxo-6H-pyrrolo[3,2,1-ij]quinoline-5-carboxamide;
(ooo) N-(4-chlorobenzyl)-1-(3-hydroxypropyl)-8-(morpholin-4-ylmethyl)-6-oxo-6H-pyrrolo[3,2,1-ij]quinoline-5-carboxamide;
(ppp) N-(4-chlorobenzyl)-1-(2-morpholin-4-ylethyl)-8-(morpholin-4-ylmethyl)-6-oxo-6H-pyrrolo[3,2,1-ij]quinoline-5-carboxamide;
(qqq) N-(4-chlorobenzyl)-8-(morpholin-4-ylmethyl)-6-oxo-1-(2-thiomorpholin-4-ylethyl)-6H-pyrrolo[3,2,1-ij]quinoline-5-carboxamide;
(rrr) N-(4-chlorobenzyl)-1-[2-(1,1-dioxidothiomorpholin-4-yl)ethyl]-8-(morpholin-4-ylmethyl)-6-oxo-6H-pyrrolo[3,2,1-ij]quinoline-5-carboxamide;
(sss) N-(4-chlorobenzyl)-1-[2-(4-methylpiperazin-1-yl)ethyl]-8-(morpholin-4-ylmethyl)-6-oxo-6H-pyrrolo[3,2,1-ij]quinoline-5-carboxamide;
(ttt) N-(4-chlorobenzyl)-8-(morpholin-4-ylmethyl)-6-oxo-1-(2-piperazin-1-ylethyl)-6H-pyrrolo[3,2,1-ij]quinoline-5-carboxamide;
(uuu) 1-[(acetylamino)methyl]-N-(4-chlorobenzyl)-8-(morpholin-4-ylmethyl)-6-oxo-6H-pyrrolo[3,2,1-ij]quinoline-5-carboxamide;
(vvv) N-(4-chlorobenzyl)-1-[(1S)-1-hydroxyethyl]-8-(morpholin-4-ylmethyl)-6-oxo-6H-pyrrolo[3,2,1-ij]quinoline-5-carboxamide;
(www) N-(4-chlorobenzyl)-1-(1H-imidazol-1-ylmethyl)-8-(morpholin-4-ylmethyl)-6-oxo-6H-pyrrolo[3,2,1-ij]quinoline-5-carboxamide;
(xxx) 1-(1H-1,2,3-benzotriazol-1-ylmethyl)-N-(4-chlorobenzyl)-8-(morpholin-4-ylmethyl)-6-oxo-6H-pyrrolo[3,2,1-ij]quinoline-5-carboxamide;
(yyy) N-(4-chlorobenzyl)-8-(morpholin-4-ylmethyl)-6-oxo-1-(pyridin-3-ylmethyl)-6H-pyrrolo[3,2,1-ij]quinoline-5-carboxamide;
(zzz) N-(4-chlorobenzyl)-8-(morpholin-4-ylmethyl)-6-oxo-1-{[({[3-(trifluoromethyl)phenyl]amino}carbonyl)amino]methyl}-6H-pyrrolo[3,2,1-ij]quinoline-5-carboxamide;
(aaaa) N-(4-chlorobenzyl)-8-(4-morpholinylmethyl)-6-oxo-2-[2-(3-oxo-1-azetidinyl)ethyl]-6H-pyrrolo[3,2,1-ij]quinoline-5-carboxamide;
(bbbb) N-(4-chlorobenzyl)-2-[2-(3-hydroxy-1-azetidinyl)ethyl]-8-(4-morpholinylmethyl)-6-oxo-6H-pyrrolo[3,2,1-ij]quinoline-5-carboxamide;
(cccc) N-(4-chlorobenzyl)-2-(2,3-dihydroxypropyl)-8-(4-morpholinylmethyl)-6-oxo-6H-pyrrolo[3,2,1-ij]quinoline-5-carboxamide;
(dddd) N-(4-chlorobenzyl)-2-[(1S)-1-hydroxyethyl]-8-(4-morpholinylmethyl)-6-oxo-6H-pyrrolo[3,2,1-ij]quinoline-5-carboxamide;
(eeee) N-(4-chlorobenzyl)-2-[2-(1H-imidazol-1-yl)ethyl]-8-(morpholin-4-ylmethyl)-6-oxo-6H-pyrrolo[3,2,1-ij]quinoline-5-carboxamide;
(ffff) N-(4-chlorobenzyl)-2-[2-(1H-imidazol-2-yl)ethyl]-8-(morpholin-4-ylmethyl)-6-oxo-6H-pyrrolo[3,2,1-ij]quinoline-5-carboxamide;
(gggg) N-(4-chlorobenzyl)-8-(morpholin-4-ylmethyl)-6-oxo-2-[2-(4H-1,2,4-triazol-3-yl)ethyl]-6H-pyrrolo[3,2,1-ij]quinoline-5-carboxamide;
(hhhh) N-(4-chlorobenzyl)-8-(morpholin-4-ylmethyl)-6-oxo-2-[2-(1H-tetraazol-5-yl)ethyl]-6H-pyrrolo[3,2,1-ij]quinoline-5-carboxamide;
(iiii) N-(4-chlorobenzyl)-8-(morpholin-4-ylmethyl)-6-oxo-2-(2-piperazin-1-ylethyl)-6H-pyrrolo[3,2,1-ij]quinoline-5-carboxamide; or
(jjjj) tert-butyl 4-{2-[5-{[(4-chlorobenzyl)amino]carbonyl}-8-(morpholin-4-ylmethyl)-6-oxo-6H-pyrrolo[3,2,1-ij]quinolin-2-yl]ethyl}piperazine-1-carboxylate, or a pharmaceutically acceptable salt thereof.

19. The method of claim 14, wherein the compound administered is N-(4-chlorobenzyl)-2-(2-hydroxyethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

20. The method of claim 14, wherein the compound administered is N-(4-chlorobenzyl)-2-(hydroxymethyl)-8-(4-morpholinylmethyl)-6-oxo-6H-pyrrolo[3,2,1-ij]quinoline-5-carboxamide, or a pharmaceutically acceptable salt thereof.

21. The method of claim 14, wherein the compound administered is N-(4-chlorobenzyl)-2-(2-hydroxyethyl)-8-(4-morpholinylmethyl)-6-oxo-6H-pyrrolo[3,2,1-ij]quinoline-5-carboxamide, or a pharmaceutically acceptable salt thereof.

22. The method of claim 14, wherein the compound administered is N-(4-chlorobenzyl)-2-(hydroxymethyl)-6-oxo-8-(tetrahydro-2H-pyran-4-ylmethyl)-6H-pyrrolo[3,2,1-ij]quinoline-5-carboxamide, or a pharmaceutically acceptable salt thereof.

23. The method of claim 1, wherein the compound is administered orally, parenterally or topically.

24. The method of claim 1, wherein the compound is administered in an amount of from about 0.1 to about 300 mg/kg of body weight.

25. The method of claim 1, wherein the compound is administered in an amount of from about 1 to about 30 mg/kg of body weight.

26. The method of claim 1, wherein said mammal is a human.

27. The method of claim 1, wherein said mammal is an animal.
